SEQUENCE LISTING (2 sequences)

SEQ ID NO: 1 - DNA, 14 bp, unidentified
  source 1..14: mol_type = unassigned DNA
ttagcacgtgctaa

SEQ ID NO: 2 - RNA, 19 nt, unidentified
  source 1..19: mol_type = unassigned RNA
nncatagcnnngctatgnn

(19) United States
(12) Patent Application Publication
(10) Pub. No.: US 2025/0154600 A1
(43) Pub. Date: May 15, 2025

(54) METHOD FOR DETECTING CANCER AND TUMOR INVASIVENESS USING DNA PALINDROMES AS A BIOMARKER

(71) Applicant: CEDARS-SINAI MEDICAL CENTER, Los Angeles, CA (US)

(72) Inventors: Hisashi TANAKA, Los Angeles, CA (US); Michael M. MURATA, Palos Verdes, CA (US); Armando E. GIULIANO, Los Angeles, CA (US)

(73) Assignee: CEDARS-SINAI MEDICAL CENTER, Los Angeles, CA (US)

(21) Appl. No.: 18/839,936

(22) PCT Filed: Mar. 6, 2023
(86) PCT No.: PCT/US2023/063761

§ 371 Date: Aug. 20, 2024

Related U.S. Application Data
(60) Provisional application No. 63/317,264, filed on Mar. 7, 2022.

(51) Int. Cl. C12Q 1/6886; C12Q 1/6806; C12Q 1/6874

(52) U.S. Cl. CPC C12Q 1/6886; C12Q 1/6874; C12Q 1/6806; C12Q 2600/112

(57) ABSTRACT

Disclosed herein are a method of detecting invasive tumor in a subject and a method of treating a subject with cancer based on invasiveness of tumor. In various embodiments, the method of detecting tumor invasiveness includes denaturing genomic DNA isolated from a tumor sample obtained from the subject; renaturing the denatured DNA for tumor DNA palindrome to form a snap back DNA; digesting the renatured DNA with a nuclease that digests single strand DNA; amplifying the tumor DNA palindrome by adapter ligation-mediated polymerase chain reaction (PCR) with genome-wide analysis of Palindrome Formation (GAPF); performing a sequence scan across multiple samples of the amplified tumor DNA palindrome; mapping reads of GAPF-seq from the sequence scan into a plurality of bins; quantifying reads in each bin; and determining whether the tumor sample is an invasive tumor based on GAPF profiles generated by analyzing the quantified reads in each bin.

### CROSS-REFERENCE TO RELATED APPLICATIONS

This application claims the benefit under 35 U.S.C. § 119(e) of U.S. provisional patent application No. 63/317,264, filed Mar. 7, 2022, the entirety of which is hereby incorporated by reference.

### STATEMENT REGARDING FEDERALLY SPONSORED RESEARCH OR DEVELOPMENT

This invention was made with government support under Grant No. W81XWH-18-1-0058 awarded by the Department of Defense and Grant No. CA149385 awarded by the National Institutes of Health. The Government has certain rights in the invention.

### REFERENCE TO SEQUENCE LISTING

This application contains a sequence listing submitted as an electronic xml file named, “Sequence_Listing_065472-000889WOPT” created on Feb. 27, 2023 (production date noted as Mar. 3, 2023) and having a size of 2,706 bytes. The information contained in this electronic file is hereby incorporated by reference in its entirety.

### FIELD

The present disclosure relates to cancer diagnostic/treatment based on palindrome profiles in samples isolated from a subject having cancer. In particular, the present disclosure relates to detecting tumor invasiveness and treating a subject with cancer based on detected tumor invasiveness.

### BACKGROUND

Cancer is diverse in character. Some of the tumors grow very slowly and never cause any harm or require treatment. Other tumors are very aggressive and grow very fast to spread in a body. For example, for diagnostic of breast cancer, mammography can catch both types of tumors. Twenty to thirty percent of tumors diagnosed by mammography are restricted, very slow-growing tumors called ductal carcinoma in situ (DCIS) or stage zero cancer. More than half of these tumors will never progress into aggressive ones in women's lifetime Thus, the majority of DCIS is not harmful. The problem is that mammography alone cannot tell which one is harmful. As a result, all the tumors diagnosed by mammography are treated equally: biopsy, surgery, chemotherapy, hormone therapy, and radiation therapy. These treatments can cause both immediate effects (breast removal, pain, and scarring, hair loss, nausea, skin burns, etc.) and long-term effects (heart disease, infertility, and secondary cancers). These treatments better be avoided if tumors are not harmful. Thus, there is a strong need to find which tumors are harmful and require intensive treatment.

Researchers and clinicians have tried to find aggressive DCIS by several recently developed genomic tests. However, these tests are not able to distinguish the aggressive DCIS from non-aggressive DCIS. Therefore, new tests and genomic markers are needed for detection of aggressive DCIS. The tests and genomic markers disclosed herein address these and other needs.

### SUMMARY OF THE INVENTION

The present disclosure provides a method of detecting invasive tumor in a subject. According to various embodiments of the present invention, the method includes denaturing genomic DNA isolated from a tumor sample obtained from the subject or denaturing cell-free DNA (cfDNA) isolated from a body fluid obtained from the subject having the tumor, to generate denatured DNA; renaturing the denatured DNA for tumor DNA palindrome to form a snap back DNA; digesting the renatured DNA with a nuclease that digests single strand DNA; amplifying the tumor DNA palindrome by adapter ligation-mediated polymerase chain reaction (PCR) with genome-wide analysis of Palindrome Formation (GAPF); performing a sequence scan across multiple samples of the amplified tumor DNA palindrome; mapping reads of GAPF-seq from the sequence scan into a plurality of bins; quantifying reads in each bin; and determining whether the tumor sample is an invasive tumor based on presence of tumor-derived DNA in the genomic DNA or cfDNA and/or GAPF profiles generated by analyzing the quantified reads in each bin.

In some embodiments, the subject is determined to have the invasive tumor when the tumor sample or body fluid is GAPF-positive or when any one of chromosomes has more than a threshold number of bins out of top 1,000 bins. In some embodiments, the subject is determined to not have an invasive tumor when the tumor sample or body fluid is GAPF-negative or when no tumor DNA palindromes are detected from the isolated genomic DNA or cfDNA. In some embodiments, the determination is based on a chromosome-specific threshold.

In some embodiments, the tumor is stage I tumor. In some embodiments, the tumor is luminal A tumor. In some embodiments, the tumor DNA palindrome clusters at CCND1 oncogene loci in the luminal A tumor. In some embodiments, the subject has breast cancer. In some embodiments, the subject has lung cancer.

In some embodiments, numbers of the GAPF-seq reads are counted for 1-kb bins. In some embodiments, top 1,000 bins are taken for analysis to determine the presence of the invasive tumor.

In some embodiments, the body fluid of the subject includes interstitial fluid, intravascular fluid, transcellular fluid, amniotic fluid, aqueous humor, bile, blood, whole blood, blood serum, blood plasma, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. In some embodiments, the body fluid includes or is blood or blood plasma.

In some embodiments, the sequence scan is a shallow scan, and the method does not require deep sequencing. In some embodiments, an amount of the genomic DNA required for generation of the GAPF profiles is about 10 ng-about 50 ng. In some embodiments, the amount of the genomic DNA is about 20 ng-about 40 ng. In some embodiments, the amount of the genomic DNA is about 25 ng-about 35 ng. In some embodiments, an amount of the genomic DNA required for generation of the GAPF profiles is about 30 ng or less.

In some embodiments, the method further includes isolating the genomic DNA from the tumor before denaturing the genomic DNA. In some embodiments, the method further includes isolating the cf DNA from the body fluid before denaturing the cfDNA. For example, the body fluid is blood plasma.

The present disclosure also provides a method of treating a subject with cancer based on invasiveness of tumor. According to various embodiments of the present invention, the method includes administering a treatment to the subject, the treatment comprising biopsy, surgery, chemotherapy, hormone therapy, and/or radiation therapy if the tumor is an invasive tumor, or the treatment comprising active monitoring, not performing biopsy, surgery, chemotherapy, hormone therapy, and radiation therapy on the subject, if the tumor is not an invasive tumor. According to various embodiments of the present invention, tumor invasiveness is detected by denaturing genomic DNA isolated from a tumor sample obtained from the subject or denaturing cell-free DNA (cfDNA) isolated from body fluid obtained from the subject having the tumor, to generate denatured DNA; renaturing the denatured DNA for tumor DNA palindrome to form a snap back DNA; digesting the renatured DNA with a nuclease that digests single strand DNA; amplifying the tumor DNA palindrome by adapter ligation-mediated polymerase chain reaction (PCR) with genome-wide analysis of Palindrome Formation (GAPF); performing a sequence scan across multiple samples of the amplified tumor DNA palindrome; mapping reads of GAPF-seq from the sequence scan into a plurality of bins; quantifying reads in each bin; and determining the invasiveness of the tumor in the subject based on presence of tumor-derived DNA in the genomic DNA or cfDNA and/or GAPF profiles generated by analyzing the quantified reads in each bin.

In some embodiments, the cancer includes breast, prostate, or lung cancer. In some embodiments, the treatment of the breast cancer includes surgery, radiation, chemotherapy, hormone therapy, targeted drug therapy, and/or immunotherapy based on a stage/type of the breast cancer if the tumor is an invasive tumor. In some embodiments, the treatment of the lung cancer includes surgery, chemotherapy, radiation therapy, targeted drug therapy, immunotherapy, palliative care, and/or alternative medicine such as acupuncture, hypnosis, massage, meditation, and yoga based on a stage/type of the lung cancer if the tumor is an invasive tumor. In some embodiments, the treatment of the prostate cancer includes surgery, radiation, cryotherapy, hormone therapy, chemotherapy, immunotherapy, and/or targeted drug therapy based on a stage/type of the prostate cancer if the tumor is an invasive tumor.

In some embodiments, the body fluid of the subject includes interstitial fluid, intravascular fluid, transcellular fluid, amniotic fluid, aqueous humor, bile, blood, whole blood, blood serum, blood plasma, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. In some embodiments, the body fluid includes blood. For example, the blood is blood plasma.

### BRIEF DESCRIPTION OF THE DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 illustrates inverted repeats (palindromes) that can fold back or snap back within strands. The following sequences are shown in FIG. 1: 5′-TTAGCACGTGCTAA-3′ (SEQ ID NO: 1) and a complementary sequence 3′-AATCGTGCACGATT-5′ (SEQ ID NO: 1), and 5′-NNCAUAGCNNNGCUAUGNN-3′ (SEQ ID NO: 2).

FIG. 2A illustrates genome-wide analysis of palindrome formation (GAPF) according to various embodiments of the present invention.

FIG. 2B illustrates an exemplary bioinformatics analysis, i.e., quantifying reads in bins according to various embodiments of the present invention.

FIGS. 3A and 3B illustrate exemplary GAPF profiles and chromosomal distributions of top 1,000 bins according to various embodiments of the present invention.

FIG. 4 illustrates separating tumor GAPF-profiles from normal GAPF-profiles according to various embodiments of the present invention.

FIG. 5A illustrates an ROC curve for breast tumor/normal classifier according to various embodiments of the present invention.

FIG. 5B illustrates an ROC curve for breast tumor/normal classifier according to various embodiments of the present invention.

FIG. 6 shows exemplary GAPF profiles in tumor stages and subtypes according to various embodiments of the present invention.

FIG. 7 shows that distributions of high coverage bins are not random.

FIG. 8 illustrates distinguishing breast Tumor DNA from normal DNA according to various embodiments of the present invention.

FIGS. 9A and 9B show that palindromes are identified at CCND1 (Cyclin D1) oncogene in several breast tumor DNA (T), but not identified in paired normal DNA (N).

FIG. 10 shows chromosomal distribution of 1,000 highest coverage bins.

FIG. 11 shows that all stage I tumors are GAPF-positive.

FIG. 12 shows subtype-specific chromosomal distribution of high coverage bins.

FIG. 13 shows a chromosome-specific threshold of more than 120 bins out of top 1,000 bins indicating that the DNA is from tumor, and thus, is GAPF-positive according to various embodiments of the present invention.

FIG. 14 shows distributions of top 1,000 1-kb bins of GAPF-seq data in two lung tumor/normal pairs.

FIG. 15 shows ROC curve and bin threshold estimate from the top 1,000 1-kb bins data from two lung normal/tumor pairs.

FIG. 16 illustrates plasma DNA/liquid biopsy analysis according to various embodiments of the present invention.

FIG. 17 illustrates liquid biopsy and breast cancer progression.

FIG. 18 shows distinguishing breast tumor DNA from normal DNA by GAPF-seq in plasma DNA/liquid biopsy analysis according to various embodiments of the present invention.

FIG. 19 shows an ROC curve for breast plasma/normal plasma classifier.

FIG. 20 shows an exemplary binary classifier based on a chromosome-specific threshold according to various embodiments of the present invention.

FIG. 21 illustrates an ROC curve of GAPF-seq profiles from 10 plasma cfDNA from cancer patients, based on each bin threshold.

FIG. 22 shows chromosomal distributions of top 1000 bins in normal (buffy coat, top) and plasma cfDNA (bottom) GAPF-seq profiles from prostate cancer patients.

FIG. 23 shows copy number profiles and tumor fractions determined by ichorCNA. 262L tumor fraction was determined using DNA extracted by phenol/chloroform (middle) and silica-beads (bottom).

FIG. 24 shows the performance of GAPF-profiles for the binary classification of breast tumor/normal DNA evaluated by machine learning algorithms, box plots (right) showing the results from three partitions.

FIG. 25 shows the performance of GAPF-profiles for the binary classification of prostate cancer patients' cfDNA/leukocyte DNA evaluated by machine learning algorithms, box plots (right) showing the results from three partitions.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.” and “for example” set off lists of one or more non-limiting examples, instances, or illustrations.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The components, steps, features, objects, benefits and advantages which have been discussed are merely illustrative. None of them, nor the discussions relating to them, are intended to limit the scope of protection in any way. Numerous other embodiments are also contemplated. These include embodiments which have fewer, additional, and/or different components, steps, features, objects, benefits and advantages. These also include embodiments in which the components and/or steps are arranged and/or ordered differently.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

All articles, patents, patent applications, and other publications that have been cited in this disclosure are incorporated herein by reference.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician. Relational terms such as “first” and “second” and the like may be used solely to distinguish one entity or action from another, without necessarily requiring or implying any actual relationship or order between them.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

Illustrative embodiments are now described. Other embodiments may be used in addition or instead. Details that may be apparent to a person of ordinary skill in the art may have been omitted. Some embodiments may be practiced with additional components or steps and/or without all of the components or steps that are described.

The details of one or more embodiments of the invention are set forth in the accompanying drawings and the description below. Other features, objects, and advantages of the invention will be apparent from the description and drawings, and from the claims.

Chromosome instability, in terms of its number and structure, is a hallmark of cancer. Gene amplification, which refers to an increase in a segmental copy number through DNA rearrangements, is an example of chromosome instability. Gene amplification is a driver of aggressive tumors, leading to overexpression of gene products and causing adverse outcomes such as oncogene amplification causing tumor progression and therapy-target gene amplification causing therapy resistance.

Although DNA palindromes are common structural aberrations, rearrangements of duplicated sequences are difficult to identify. We show that genome-wide analysis of Palindrome Formation (GAPF) can be used as a test to determine invasiveness of a tumor sample. Since aggressive tumors are positive for the test, harmful DCIS could also be positive, while harmless ones would be negative for the test. Thus, our genomic test disclosed herein is used to detect harmful DCIS and prevent unnecessary treatments for harmless DCIS.

Disclosed herein is a method of detecting a genome-wide analysis of Palindrome Formation (GAPF) profile in a tumor sample obtained from a subject in need thereof. According to various embodiments of the present disclosure, the method includes denaturing genomic DNA isolated from a tumor sample obtained from the subject; renaturing the denatured DNA for tumor DNA palindrome to form a snap back DNA; digesting the renatured DNA with a nuclease that digests single strand DNA; amplifying the tumor DNA palindrome by adapter ligation-mediated polymerase chain reaction (PCR) with genome-wide analysis of Palindrome Formation (GAPF); performing a sequence scan across multiple samples of the amplified tumor DNA palindrome; mapping reads of GAPF-seq from the sequence scan into a plurality of bins; quantifying reads in each bin; and detecting the GAPF profiles generated by analyzing the quantified reads in each bin. According to various embodiments of the present disclosure, the method further includes isolating the genomic DNA to be denatured from the subject.

According to various embodiments of the present disclosure, the tumor sample is GAPF-positive or when any one of chromosomes has more than a threshold number of bins out of top 1,000 bins; or the tumor sample is GAPF-negative or when no tumor DNA palindromes are detected from the isolated genomic DNA. In some embodiments, the threshold number of bins is 120. In some embodiments, the determination is based on a chromosome-specific threshold.

Disclosed herein is a method of detecting tumor invasiveness in a subject. According to various embodiments of the present disclosure, the method includes denaturing genomic DNA isolated from a tumor sample obtained from the subject; renaturing the denatured DNA for tumor DNA palindrome to form a snap back DNA; digesting the renatured DNA with a nuclease that digests single strand DNA; amplifying the tumor DNA palindrome by adapter ligation-mediated polymerase chain reaction (PCR) with genome-wide analysis of Palindrome Formation (GAPF); performing a sequence scan across multiple samples of the amplified tumor DNA palindrome; mapping reads of GAPF-seq from the sequence scan into a plurality of bins; quantifying reads in each bin; and determining whether the tumor sample is an invasive tumor based on GAPF profiles generated by analyzing the quantified reads in each bin. According to various embodiments of the present disclosure, the method further includes isolating the genomic DNA to be denatured from the subject.

According to various embodiments of the present disclosure, DNA is fragmented by restriction enzyme digestion. To fragment DNA, DNA is mixed with nuclease-free H₂O (for example, 30-1,000 ng of DNA is mixed with nuclease-free H₂O to a total volume of 34 μL in a 1.7 mL microcentrifuge tube); in a new microcentrifuge tube, the DNA solution is mixed with KpnI (10 U), and NEBuffer 1.1 (for example, in a new 1.7 mL microcentrifuge tube, 17 μL of the DNA solution is mixed with 1 μL KpnI (10 U), and 2 μL 10× NEBuffer 1.1 for a total volume of 20 μL); in a microcentrifuge tube, the remaining DNA solution is mixed with SbfI (10 U), and CutSmart buffer (for example, in a new 1.7 mL microcentrifuge tube, the remaining 17 μL of the DNA solution is mixed with 1 μL SbfI (10 U), and 2 μL CutSmart buffer for a total volume of 20 μL), incubate at 37° C. in a water bath overnight or more than 16 hours; briefly spin in a microcentrifuge to bring the liquid to the bottom; and heat at 65° C. for 20 minutes to inactivate restriction enzymes.

According to various embodiments of the present disclosure, snap-back is performed by briefly spinning in a microcentrifuge to bring the liquid to the bottom; mixing the KpnI-digested DNA (for example, 20 μL) and SbfI-digested DNA (for example, 20 μL) with 5M NaCl, formamide, and nuclease-free H₂O in a thin-wall PCR tube; (for example, 1.8 μL 5M NaCl, 45 μL formamide, and 3.2 μL nuclease-free H₂O are mixed in a thin-wall PCR tube); applying a cap lock to prevent the tube from opening during DNA denaturing; heating the DNA mixture in boiling water for several minutes, for example, 7 or about 7 minutes, to denature DNA; and immediately quenching the DNA mixture in ice water for several minutes, for example, 5 or about 5 minutes, to rapidly renature DNA.

According to various embodiments of the present disclosure, S1 digestion is performed by briefly spinning in a microcentrifuge to bring the liquid to the bottom; adding 5M NaCl, 10× S1 nuclease buffer, S1 nuclease (20 U/μL), and nuclease-free H₂O to the DNA mixture (for example, 4.8 μL 5M NaCl, 12 μL 10× S1 nuclease buffer, 2 μL S1 nuclease (20 U/μL), and 11.2 μL nuclease-free H₂O are added to the DNA mixture); and incubating at 37° C. in a water bath for 1 or about 1 hour.

According to various embodiments of the present disclosure, DNA is purified, for example, using Monarch PCR and DNA Clean-up Kit. The following protocol is performed to purify DNA: centrifugation, for example, at 16,000×g (˜13,000 rpm), at room temperature; add DNA Cleanup Binding Buffer (for example, 240 μL) to the S1 digested-DNA sample; mix well (for example, by pipetting 10 times); briefly spin in a microcentrifuge to bring the liquid to the bottom; move liquid to a column, insert column into a collection tube (for example, 2 mL collection tube), and close the cap; centrifuge for 1 minute and then discard the flow-through; add DNA Wash Buffer (for example, 200 μL), centrifuge for 1 minute, and then discard the flow-through (for example, repeat this step once); insert the empty column into the collection tube and centrifuge for 1 minute; transfer the column to a new collection tube; add DNA Elution Buffer (for example, 15 μL) and incubate for 1 minute at room temperature; centrifuge, for example, for 1 minute; add DNA Elution Buffer (for example, 10 μL) and incubate, for example, for 1 minute, at room temperature; and centrifuge, for example, for 1 minute, and save the sample.

According to various embodiments of the present disclosure, Library Construction is performed, for example, using NEBNext Ultra II FS DNA Library Prep Kit for Illumina. The following protocol is performed for Library Construction: mix DNA, nuclease-free H₂O, NEBNext Ultra II FS Reaction Buffer, and NEBNext Ultra II FS Enzyme Mix in a PCR tube (for example, mix 22 μL of DNA, 4 μL nuclease-free H₂O, 7 μL NEBNext Ultra II FS Reaction Buffer, and 2 μL NEBNext Ultra II FS Enzyme Mix are mixed in the PCR tube); vortex reaction briefly, for example, for 5 seconds, and briefly spin in a centrifuge to bring the liquid to the bottom; in a thermocycler with the lid heated to 75° C., incubate the reaction, for example, for 15 minutes, at 37° C. followed by 30 minutes at 65° C. and then held at 4° C.; add to the reaction mixture Ligation Enhancer, diluted NEBNext Adaptor, and Ligation Master Mix (for example, 1 μL Ligation Enhancer, 2.5 μL diluted NEBNext Adaptor, and 30 μL Ligation Master Mix are add to the reaction mixture); mix well, for example, by pipetting 10 times set to 50 L, and briefly spin in a microcentrifuge to bring the liquid to the bottom; in a thermocycler with no heated lid, incubate the reaction, for example, for 15 minutes, at 20° C. and then held at 4° C.; add to the reaction mixture 3 μL USER Enzyme; mix well, for example, by pipetting 10 times set to 50 μL, and briefly spin in a microcentrifuge to bring the liquid to the bottom; in a thermocycler with the lid heated to at least 47° C., incubate the reaction, for example, for 15 minutes, at 37° C. and then held at 4° C.; vortex magnetic beads; add magnetic beads (for example, 57 μL) to adaptor-ligated DNA; incubate at room temperature, for example, for 5 minutes; place magnetic bead-DNA mixture on magnet for 5 or about 5 minutes; remove supernatant; on magnet, add 80% ethanol (for example, 200 μL), wait for 30 or about 30 seconds, and then remove supernatant (repeat this step once); air dry the magnetic beads, for example, for 3 minutes; off magnet, add 0.1× low TE buffer (for example, 17 μL); mix well, for example, by pipetting 10 times; incubate at room temperature for 5 or about 5 minutes; place magnetic bead-DNA mixture on magnet for 5 or about 5 minutes; remove 15 μL supernatant and put into a new PCR tube; add 5 μL Universal PCR Primer, 5 μL Index Primer, and 25 μL NEBNext Q5 Master Mix; mix well, for example, by pipetting 10 times set to 40 μL, and briefly spin in a microcentrifuge to bring the liquid to the bottom; in a thermocycler with the lid heated to at least 103° C., incubate the reaction for 30 seconds at 98° C. followed by 20 cycles of 10 seconds at 98° C. and 75 seconds at 65° C., then 5 minutes at 65° C. and held at 4° C.; vortex magnetic beads; add 45 μL magnetic beads to adaptor-ligated DNA; incubate at room temperature for 5 minutes; place magnetic bead-DNA mixture on magnet for 5 minutes; remove supernatant; on magnet, add 80% ethanol (for example, 200 μL), wait for 30 seconds, and then remove supernatant (repeat this step once); air dry the magnetic beads for 3 or about 3 minutes; off magnet, add 0.1× low TE buffer (for example, 33 μL); mix well, for example, by pipetting 10 times; incubate at room temperature for 5 or about 5 minutes; place magnetic bead-DNA mixture on magnet for 5 or about 5 minutes; remove 30 μL supernatant and store in a DNA LoBind tube; measure concentration of DNA with High Sensitivity Qubit Fluorometer for dsDNA using 2 μL of sample; check size distribution, for example, using Agilent Bioanalyzer High Sensitivity DNA chip; and sequence samples, for example, using an Illumina-based sequencing platform with low sequencing depth (0.5-1.0× coverage is sufficient).

According to various embodiments of the present disclosure, data analysis is performed as follows: trim raw *.fastq data with Trim galore (v0.6.1) and Cutadapt (v2.3) with parameters ‘--length 55’; align trimmed *.fastq data to hg38 reference genome using Bowtie2 (v2.3.5) with unpaired alignment; convert *.sam alignment file using Samtools (v1.9) to binary format and sort the subsequent *.bam files; filter uniquely mapped reads by applying a mapping quality filter of 40 μsing the ‘samtools view’ command with parameters ‘-b -q 40’; extract the number of sequencing reads after applying the mapped quality filter to determine the per million scaling factor to normalize for mapping depth; sort *.bam file using Samtools and convert to *.bed format using Bedtools (v2.28.0); Sort *.bed files using the ‘sort’ command with parameters ‘-k1,1 -k2,2n’; use Bedtools2 to take an alignment of reads as input and generate a coverage track as output in 1 kb non-overlapping bins with parameters ‘-sorted -counts’; use the scaling factor to normalize the coverage in 1 kb bins for the mapping depth; and locate regions of high coverage bins to identify de novo DNA palindromes.

According to various embodiments of the present disclosure, the subject is determined to have the invasive tumor when the tumor sample is GAPF-positive or when any one of chromosomes has more than a threshold number of bins out of top 1,000 bins; or the subject is determined to not have an invasive tumor when the tumor sample is GAPF-negative or when no tumor DNA palindromes are detected from the isolated genomic DNA. In some embodiments, the threshold number of bins is 120. In some embodiments, the determination is based on a chromosome-specific threshold.

According to various embodiments of the present disclosure, the tumor is stage I tumor. In some embodiments, the tumor is luminal A tumor. In some embodiments, the tumor DNA palindrome clusters at CCND1 oncogene loci in the luminal A tumor.

According to various embodiments of the present disclosure, the subject has breast cancer. According to various embodiments of the present disclosure, the subject has lung cancer. According to various embodiments of the present disclosure, the subject has prostate cancer. However, the types of cancer are not limited to the breast cancer, lung cancer, and prostate cancer; for example, the type of cancer can further include bladder cancer, cervical cancer, colorectal cancer, gynecologic cancers including cervical, ovarian, uterine, vaginal, and vulvar, head and neck cancers, kidney cancer, liver cancer, lymphoma, mesothelioma, myeloma, ovarian cancer, skin cancer, thyroid cancer, uterine cancer, and vaginal and vulvar cancers among others.

In some embodiments, numbers of the GAPF-seq reads are counted for 1-kb bins. For example, top 1,000 bins are taken for analysis to determine the presence of the invasive tumor.

In some embodiments, instead of isolating genomic DNA from the tumor sample, the genomic DNA is isolated from body fluid of the subject. For example, the body fluid includes interstitial fluid, intravascular fluid, transcellular fluid, amniotic fluid, aqueous humor, bile, blood, whole blood, blood serum, blood plasma, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. For example, the body fluid includes blood. For example, the body fluid includes blood plasma.

In some embodiments, instead of isolating genomic DNA from the tumor sample or body fluid, cell-free DNA (cfDNA) is isolated from body fluid of the subject. For example, the body fluid includes interstitial fluid, intravascular fluid, transcellular fluid, amniotic fluid, aqueous humor, bile, blood, whole blood, blood serum, blood plasma, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. For example, the body fluid includes blood. For example, the body fluid includes blood plasma.

In some embodiments, the sequence scan is a shallow scan, and the method does not require deep sequencing. In some embodiments, an amount of the genomic DNA required for generation of the GAPF profiles is about 10 ng-about 50 ng. For example, the amount of the genomic DNA is about 20 ng-about 40 ng. For example, the amount of the genomic DNA is about 25 ng-about 35 ng. In some embodiments, an amount of the genomic DNA required for generation of the GAPF profiles is about 30 ng or less.

Disclosed herein is a method of detecting tumor invasiveness in a subject. According to various embodiments of the present disclosure, the method includes denaturing genomic DNA isolated from a tumor sample obtained from the subject or denaturing cell-free DNA (cfDNA) isolated from body fluid obtained from the subject having the tumor, to generate denatured DNA; renaturing the denatured DNA for tumor DNA palindrome to form a snap back DNA; digesting the renatured DNA with a nuclease that digests single strand DNA; amplifying the tumor DNA palindrome by adapter ligation-mediated polymerase chain reaction (PCR) with genome-wide analysis of Palindrome Formation (GAPF); performing a sequence scan across multiple samples of the amplified tumor DNA palindrome; mapping reads of GAPF-seq from the sequence scan into a plurality of bins; quantifying reads in each bin; and determining the invasiveness of the tumor in the subject based on presence of tumor-derived DNA in the genomic DNA or cfDNA and/or GAPF profiles generated by analyzing the quantified reads in each bin. For example, the cancer includes breast, prostate, or lung cancer.

Disclosed herein is a method of detecting invasive tumor in a subject and treating the subject. According to various embodiments of the present disclosure, the method includes denaturing genomic DNA isolated from a tumor sample obtained from the subject or denaturing cell-free DNA (cfDNA) isolated from body fluid obtained from the subject having the tumor, to generate denatured DNA; renaturing the denatured DNA for tumor DNA palindrome to form a snap back DNA; digesting the renatured DNA with a nuclease that digests single strand DNA; amplifying the tumor DNA palindrome by adapter ligation-mediated polymerase chain reaction (PCR) with genome-wide analysis of Palindrome Formation (GAPF); performing a sequence scan across multiple samples of the amplified tumor DNA palindrome; mapping reads of GAPF-seq from the sequence scan into a plurality of bins; quantifying reads in each bin; determining the invasiveness of the tumor in the subject based on presence of tumor-derived DNA in the genomic DNA or cfDNA and/or GAPF profiles generated by analyzing the quantified reads in each bin; and administering a treatment to the subject, the treatment comprising biopsy, surgery, chemotherapy, hormone therapy, and/or radiation therapy if the tumor is an invasive tumor or the treatment comprising active monitoring, not performing biopsy, surgery, chemotherapy, hormone therapy, and radiation therapy on the subject, if the tumor is not an invasive tumor.

According to various embodiments of present disclosure, the treatment for the subject having breast cancer includes surgery, radiation, chemotherapy, hormone therapy, targeted drug therapy, and/or immunotherapy based on the stage/type of the breast cancer. According to various embodiments of present disclosure, the treatment for the subject having lung cancer includes surgery, chemotherapy, radiation therapy, targeted drug therapy, immunotherapy, palliative care, and/or alternative medicine such as acupuncture, hypnosis, massage, meditation, and yoga based on the stage/type of the lung cancer. According to various embodiments of present disclosure, the treatment for the subject having prostate cancer includes surgery, radiation, cryotherapy, hormone therapy, chemotherapy, immunotherapy, and/or targeted drug therapy based on the stage/type of the prostate cancer.

Also disclosed herein is a method treating the subject. According to various embodiments of the present disclosure, the method includes request the results regarding a detection invasive cancer, the detection method comprising denaturing genomic DNA isolated from a tumor sample obtained from the subject or denaturing cell-free DNA (cfDNA) isolated from body fluid obtained from the subject having the tumor, to generate denatured DNA; renaturing the denatured DNA for tumor DNA palindrome to form a snap back DNA; digesting the renatured DNA with a nuclease that digests single strand DNA; amplifying the tumor DNA palindrome by adapter ligation-mediated polymerase chain reaction (PCR) with genome-wide analysis of Palindrome Formation (GAPF); performing a sequence scan across multiple samples of the amplified tumor DNA palindrome; mapping reads of GAPF-seq from the sequence scan into a plurality of bins; quantifying reads in each bin; determining the invasiveness of the tumor in the subject based on presence of tumor-derived DNA in the genomic DNA or cfDNA and/or GAPF profiles generated by analyzing the quantified reads in each bin; and administering a treatment to the subject, the treatment comprising biopsy, surgery, chemotherapy, hormone therapy, and/or radiation therapy if the tumor is an invasive tumor, or the treatment comprising active monitoring, not performing biopsy, surgery, chemotherapy, hormone therapy, and radiation therapy on the subject, if the tumor is not an invasive tumor.

Disclosed herein is a method of treating a subject with cancer based on invasiveness of tumor. According to various embodiments of the present disclosure, the method includes administering a treatment to the subject, the treatment comprising biopsy, surgery, chemotherapy, hormone therapy, and/or radiation therapy if the tumor is an invasive tumor; or with the treatment comprising active monitoring, not performing biopsy, surgery, chemotherapy, hormone therapy, and radiation therapy on the subject, if the tumor is not an invasive tumor. According to various embodiments of the present disclosure, invasiveness of the tumor is detected by denaturing genomic DNA isolated from a tumor sample obtained from the subject or denaturing cell-free DNA isolated from body fluid obtained from the subject having the tumor, to generate denatured DNA; renaturing the denatured DNA for tumor DNA palindrome to form a snap back DNA; digesting the renatured DNA with a nuclease that digests single strand DNA; amplifying the tumor DNA palindrome by adapter ligation-mediated polymerase chain reaction (PCR) with genome-wide analysis of Palindrome Formation (GAPF); performing a sequence scan across multiple samples of the amplified tumor DNA palindrome; mapping reads of GAPF-seq from the sequence scan into a plurality of bins; quantifying reads in each bin; and determining the invasiveness of the tumor in the subject based on presence of tumor-derived DNA in the genomic DNA or cfDNA and/or GAPF profiles generated by analyzing the quantified reads in each bin.

Palindrome profiles have never been considered for cancer diagnostics. Palindrome profiles could differentiate aggressive tumors from indolent tumors or normal tissues. However, palindromes are challenging to study. For example, palindromes cannot be amplified by general polymerase chain reaction (PCR), as Taq polymerases cannot navigate the secondary structure of self-annealed palindromes. Therefore, any technologies involving PCR, including (the library construction step of) Whole Genome Sequencing (WGS), could suffer from the underrepresentation of DNA palindromes. Genome-wide analysis of palindrome formation (GAPF-seq) takes advantage of the self-annealing propensity of a palindrome. Once folding back, palindromes lose secondary structures and can be amplified by PCR and overcomes these problems.

In this regard, we have developed a genomic test that utilizes abnormal DNA structure, i.e., DNA palindromes to detect cancer and the aggressiveness of cancer. DNA palindrome is a DNA sequence that reads the same backward as forward, and very often present in cancer DNA. According to our investigation of DNA palindromes by the inventive genomic test, almost all the aggressive cancer was positive for the test, while normal DNA was negative for the test. Surprisingly, although DCIS is considered cancer, DCIS was negative for the test.

Benefits of our palindrome detection method for a cancer detection test include but are not limited to the following.
1. GAPF-seq is relatively simple and only requires genomic DNA, and scan through the entire genome for DNA palindromes.
2. Less than 100 ng or 30 ng of genomic DNA is sufficient to produce reproducible GAPF profiles. The assay can be further optimized for very small tissue samples.
3. Palindrome detection in 25-fold excess of non-palindromic DNA was previously shown in Tanaka et al. Nature Genetics 37, 320-327 (2005). Thus, genome-wide analysis of palindrome formation is performed to identify structural chromosome aberrations associated with cancer.
4. Because GAPF enriches a particular feature in the genome, it does not require ultra-deep sequencing, which could circumvent the emerging problem of big data storage.

WGS can detect palindromes (fold-back inversions). However, as mentioned above, palindromes can be underrepresented due to technical difficulties. Also, to identify palindromes in WGS, very deep sequencing is necessary. Very deep sequencing, i.e., sequencing a genomic region multiple times, requires a lot of data and high cost, and is not feasible for cancer detection test. Thus, we are enriching DNA palindromes, one of the most common features of cancer genome aberrations, using our own unique methods.

Disclosed herein is genomic approach for a genome-wide analysis of palindrome formation (GAPF-seq). GAPF-seq scans through individual tumor genomes for aberrant DNA structures (DNA palindromes, also called fold-back inversions), which are DNA sequences that read the same backward as forward. We have shown that DNA palindromes arise from common adverse events causing cancer genome instability, such as illegitimate repair of chromosome breaks and telomere dysfunction. Our genomic studies have demonstrated that GAPF-seq can locate DNA palindromes in cancer genomes, which often demarcate oncogene amplification.

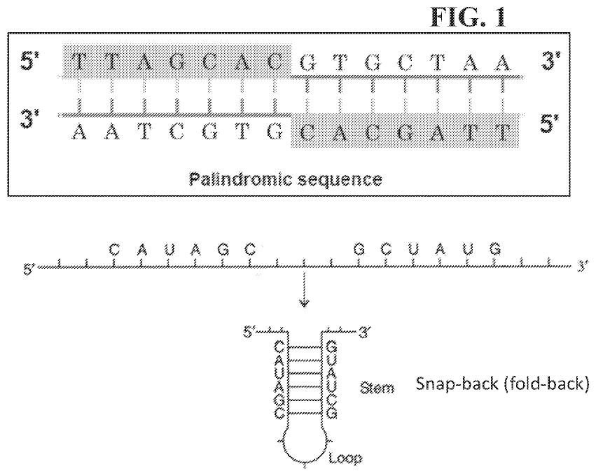

GAPF-seq exploits the propensity of denatured DNA palindromes to form double-stranded DNA (dsDNA) by intra-molecular annealing. Referring to FIG. 1, denatured palindromes or inverted repeats can fold back (snap back) within the strands when renatured. FIG. 1 shows a double-stranded nucleotide having a sequence of 5′-TTAGCACGTGCTAA-3′ (SEQ ID NO: 1) and a complementary sequence 3′-AATCGTGCACGATT-5′ (SEQ ID NO: 1). These sequences are palindromic since reading in a certain direction (e.g. 5′ to 3′) on one strand is identical to the sequence in the same direction (e.g. 5′ to 3′) on the complementary strand. Further, if the double-stranded nucleotide is denatured, each single-stranded nucleotide is a palindrome. For example, the nucleotide sequence TTAGCACGTGCTAA (SEQ ID NO: 1) is palindromic with its nucleotide-by-nucleotide complement AATCGTGCACGATT (SEQ ID NO: 1) because reversing the order of the nucleotides in the complement gives the original sequence. FIG. 1 also shows an example of a single stranded nucleotide NNCAUAGCNNNGCUAUGNN (SEQ ID NO: 2) including a first sequence CAUAGC and a second sequence GCUAUG, additional nucleotide bases being present between the first sequence and the second sequence. Since this is a palindromic nucleotide sequence it is capable of forming a loop having a hairpin structure by snap back, as shown in FIG. 1.

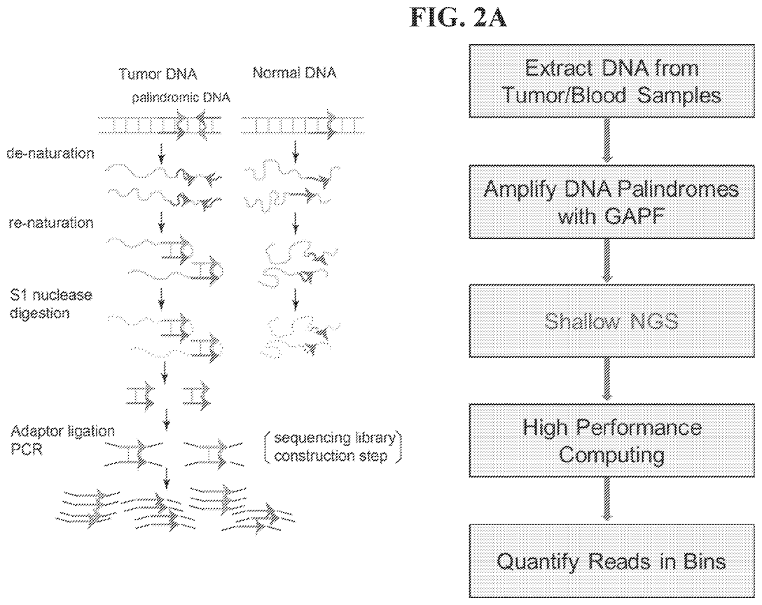

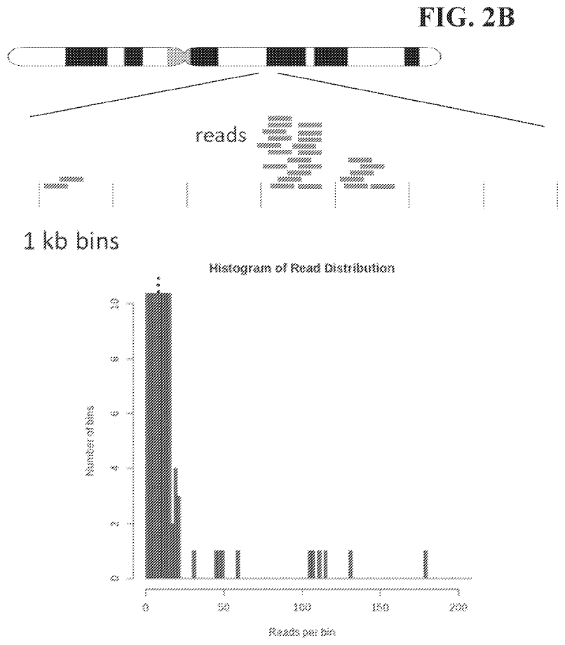

Referring to FIG. 2A, DNA samples are extracted from tumor/blood samples. To enrich DNA palindromes, genomic DNA is denatured and quickly renatured to favor intra-molecular annealing under conditions that do not favor intermolecular annealing. The remaining single-stranded, non-palindromic DNA is digested by single-strand-specific nuclease S1. The dsDNA from palindromes is concurrently amplified by the process of constructing libraries for next-generation sequencing (NGS), by which DNA palindromes appear as coverage peaks in the genome. The relatively simple GAPF-seq procedure simultaneously amplifies the target signal and reduces background noise. Shallow Whole Genome Sequencing (shallow WGS, also known as low pass whole genome sequencing or NGS), which is a massively parallel sequencing technology that offers ultra-high throughput, scalability, and speed, is used to achieve genome-wide genetic variation accurately and cost-effectively.

By employing high performance computing, sequence reads are quantified in a plurality of bins, as shown in FIG. 2B. Each respective bin in the plurality of bins represents a different portion of the DNA sample or genomic DNA. For each respective bin in the plurality of bins, there is a set of sequence reads in a plurality of sets of sequence reads. Each sequence read in each set of sequence reads in the plurality of sets of sequence reads is in the plurality of sequence reads. For example, the size of each bin is 1 kb, 2 kb, 3 kb, 4 kb, or 5 kb such that sequencing reads are mapped in 1-kb bins, 2-kb bins, 3-kb bins, 4-kb bins, or 5-kb bins, respectively. In particular embodiments, the size of each bin is about 1 kb such that sequencing reads are mapped in 1-kb bins. In some embodiments, bins are overlapping, a sequence read in one bin at least partially overlapping a sequence read in another bin. In some embodiments, the bins are non-overlapping. We used an algorithm for identifying bins with a high number of sequence reads throughout the entire genome and generating a palindrome profile for the sample. The likelihood of having palindromes is represented by the depth of sequencing reads. The number of sequence reads within each bin was divided by a per-million scaling factor (for example, the scaling factor is 100 for a run with 100 million reads) in order to adjust for the total sequencing depth of a particular sequencing run (adjusted read coverage, ARC). Bins containing high ARC will demarcate DNA palindromes.

For example, we divided the entire genome into 3 million 1-kb bins, each 1-kb bin having a unique genomic sequence. Reads from GAPF-seq were assigned to 1-kb bins according to the sequence composition of reads. Bins assigned with a very high number of bins were likely to have palindromes.

De novo palindromes formed by this mechanism can span several million base pairs. Prior to denaturation/renaturation, we digested tumor DNA containing such palindromes by rare-cutting restriction enzymes, and dsDNA after renaturation only forms from the DNA that contains the centers of palindromes. Since the DNA fragments are ranging from a few kb to 20 kb, we expect dsDNA after denaturation and quick renaturation to be less than 10 kb. Therefore, our algorithm considers five consecutive 1 kb bins with an ARC>1.5. Normalizing to sequencing depth uses a “per million” scaling factor where the number of reads in each bin is divided by the total number of millions of reads (e.g., a scaling factor of 20 for 20 million reads) Adjusted read coverage (ARC)>1.5. For palindromes, five (5) consecutive bins need to be enriched above this threshold (i.e., the palindrome must span 5 kb).

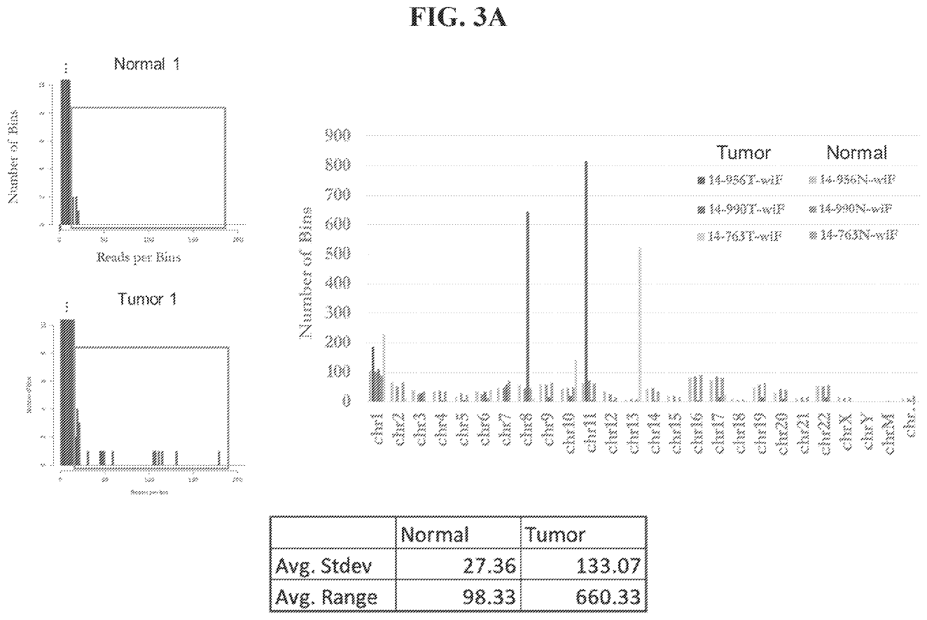

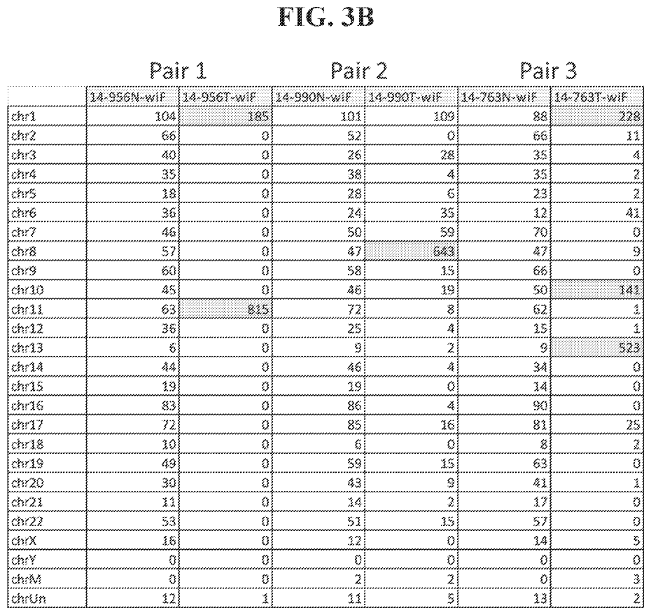

FIGS. 3A and 3B show exemplary GAPF profiles. In FIG. 3A, chromosomal distributions of top 1,000 bins are profiled for normal DNA and tumor DNA. According to FIG. 3B, GAPF profiles show tumor-specific clustering.

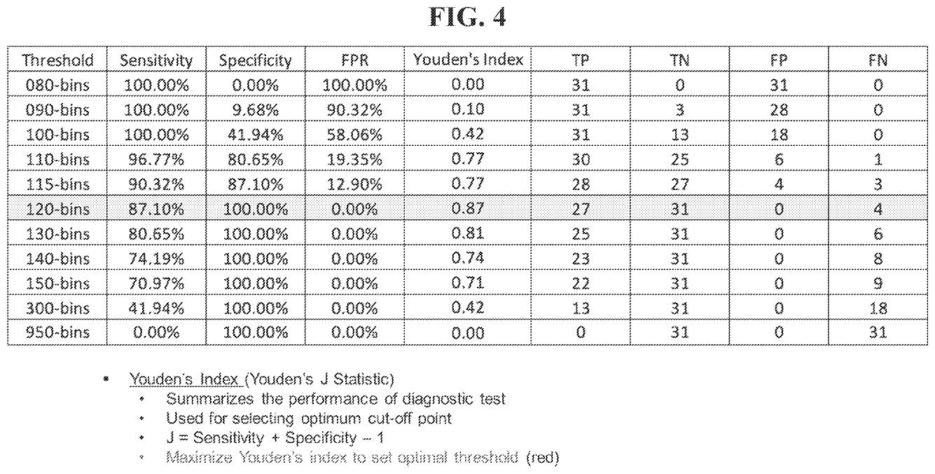

FIG. 4 shows that tumor GAPF profiles can be separated from normal GAPF profiles.

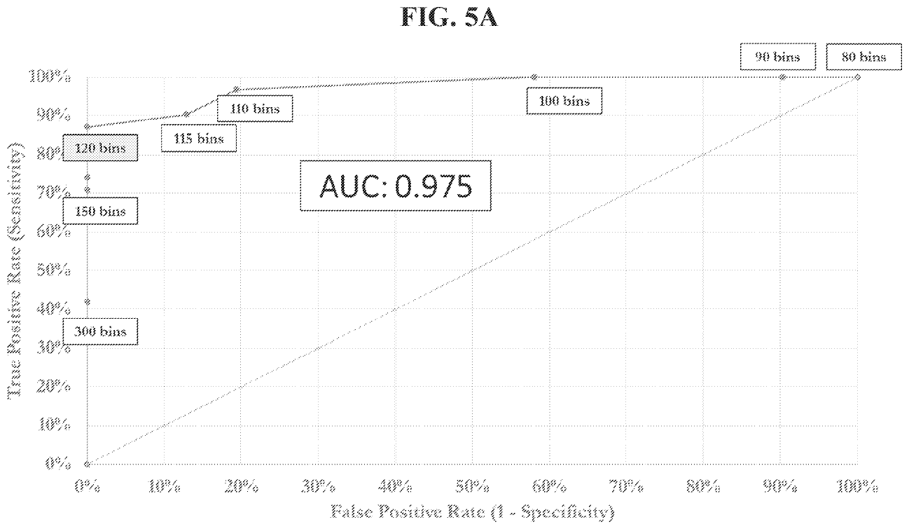

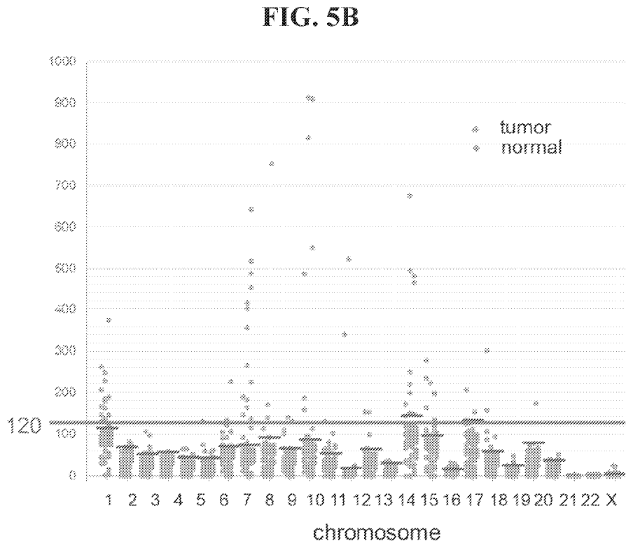

FIG. 5A shows Receiver Operating Characteristic (ROC) curve for breast tumor/normal classifier. The ROC curve illustrates diagnostic ability of binary classifier system (e.g., tumor vs. normal) and plots true positive rate (TPR) (sensitivity) against false positive rate (FPR) (1-specificity) with varying thresholds. Area under the ROC Curve (AUC) is calculated by trapezoidal rule. Referring to FIG. 5B, if any one of the chromosomes has more than 120 bins out of top 1,000 bins, the DNA is from tumor, and thus, is GAPF-positive.

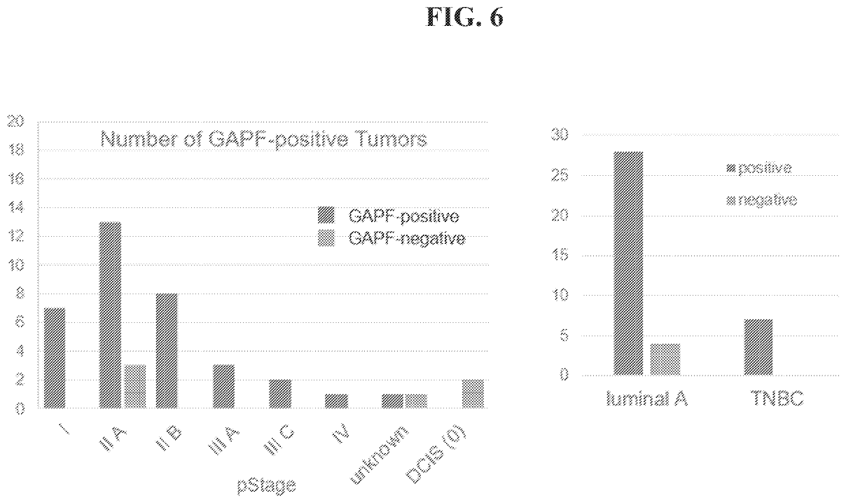

FIG. 6 shows GAPF profiles in tumor stages and subtypes. All six stage I tumors are GAPF-positive, indicating that palindrome formation (inverted duplication) is an early event of tumor development.

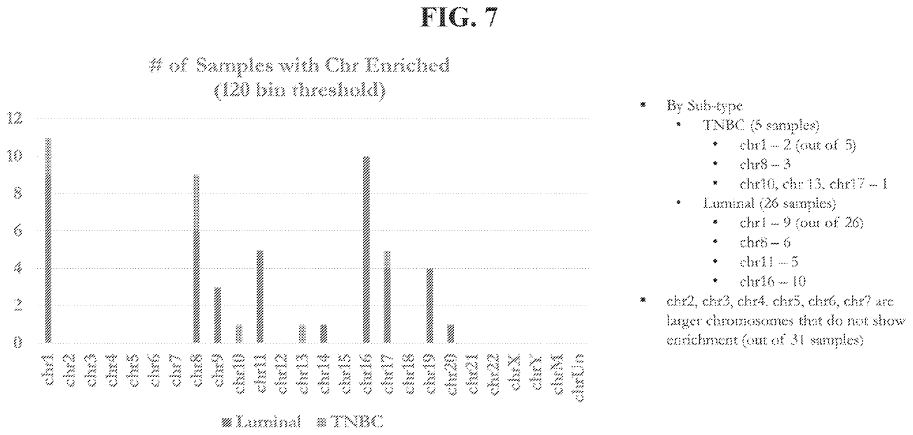

FIG. 7 shows that distributions of high coverage bins are not random.

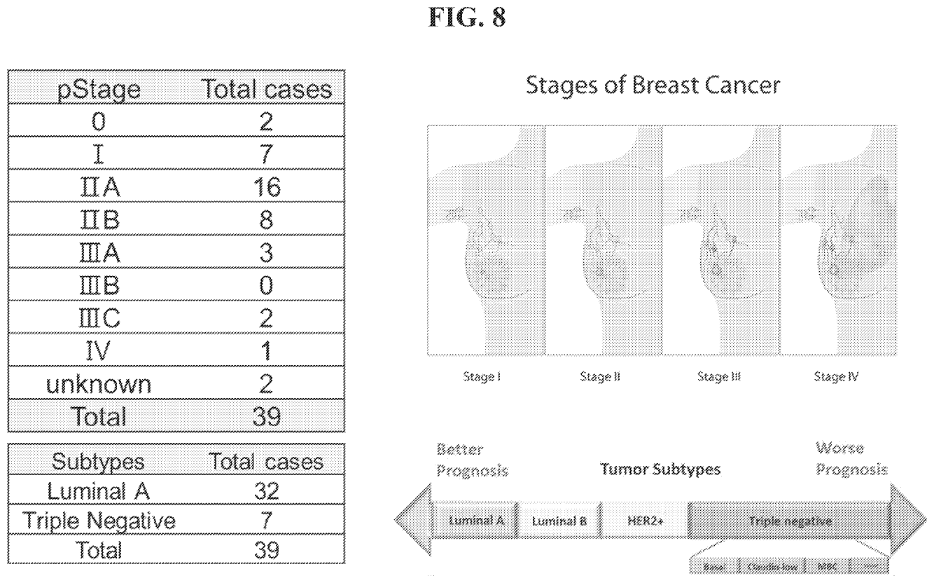

Referring to FIG. 8, we have developed a cancer detection pipeline using the GAPF-seq data from 39 inflammatory breast cancer (IBC)/normal pairs and showed that GAPF-seq could effectively differentiate IBC DNA, including all stage I IBC DNA, from paired normal DNA (sensitivity 89.7% and specificity 100%, see FIG. 5A.). This study was done using breast cancer tissue biopsy.

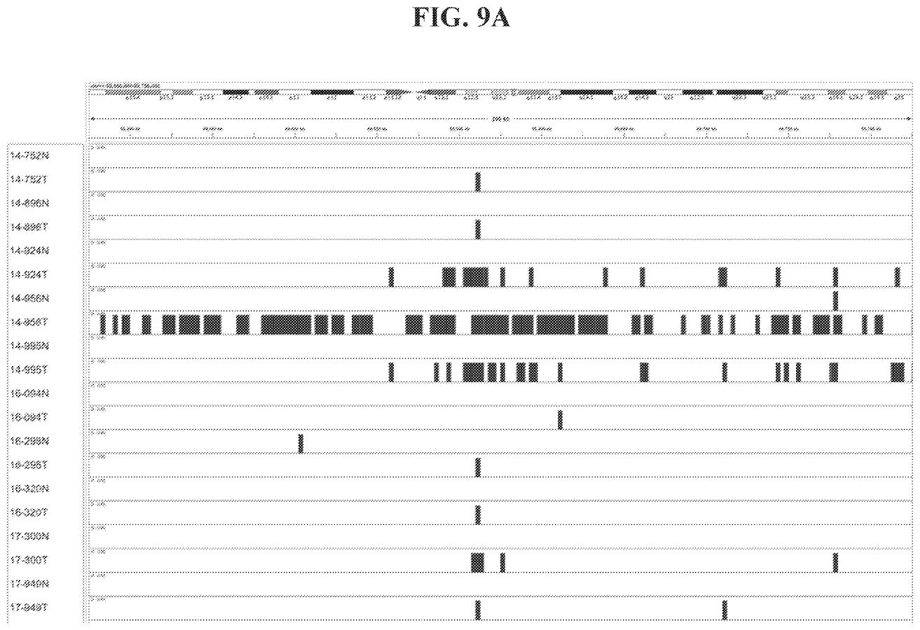

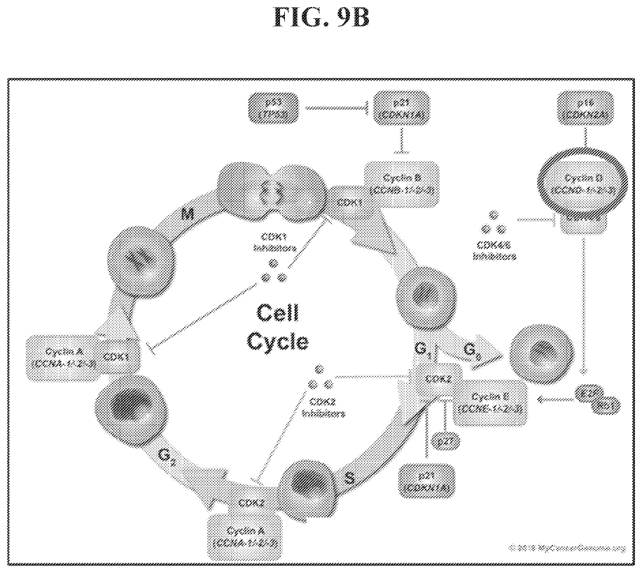

Referring to FIG. 9A, we found that DNA palindromes are non-randomly distributed and cluster at CCND1 (Cyclin D1) oncogene loci in luminal A tumors. According to the data from the normal/tumor pairs, palindromes are identified at CCND1 oncogene in several breast tumors (T). However, normal samples (N) did not have any palindromes. FIG. 9B illustrates a cell cycle involving various Cyclins.

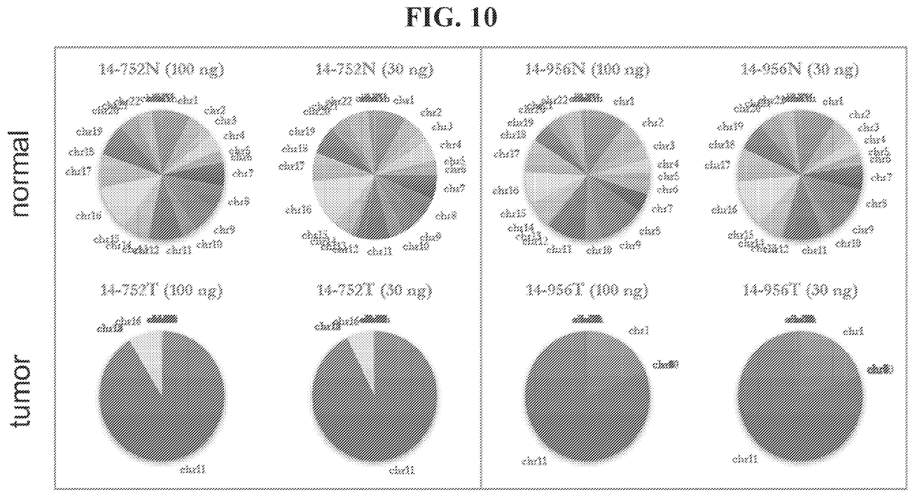

FIG. 10 shows chromosomal distribution of 1,000 highest coverage bins. Skewed chromosomal distributions are present in tumors. Further, GAPF profiles are highly reproducible between duplicates. For example, the results can be reproduced using 30 ng of input DNA

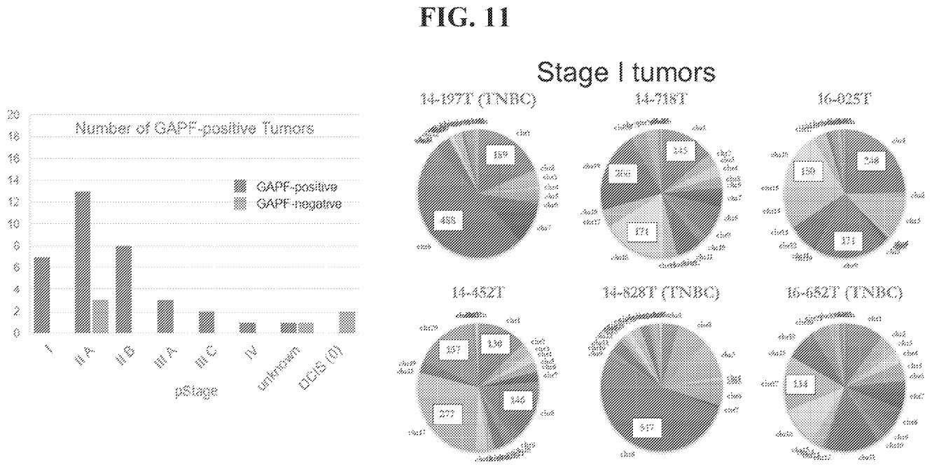

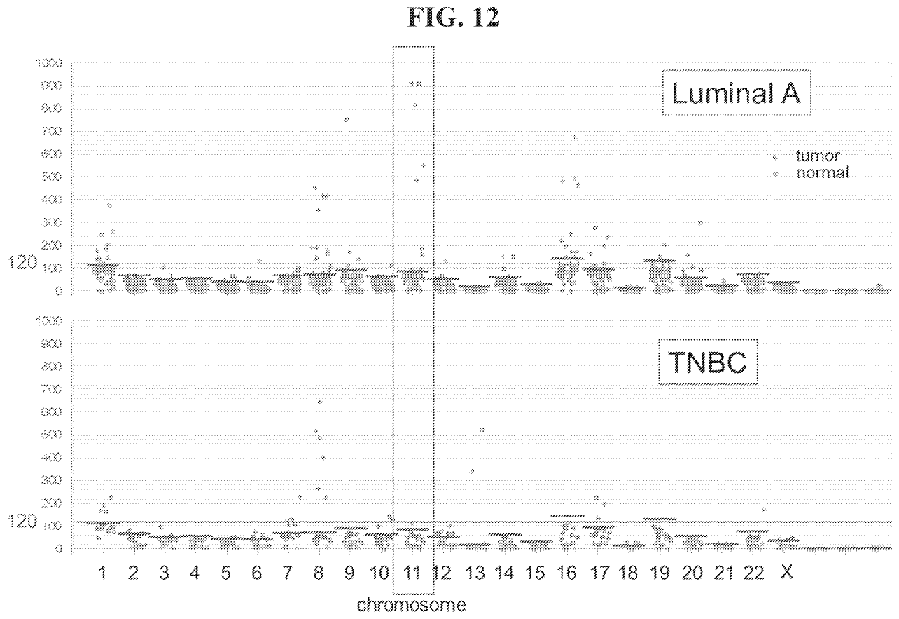

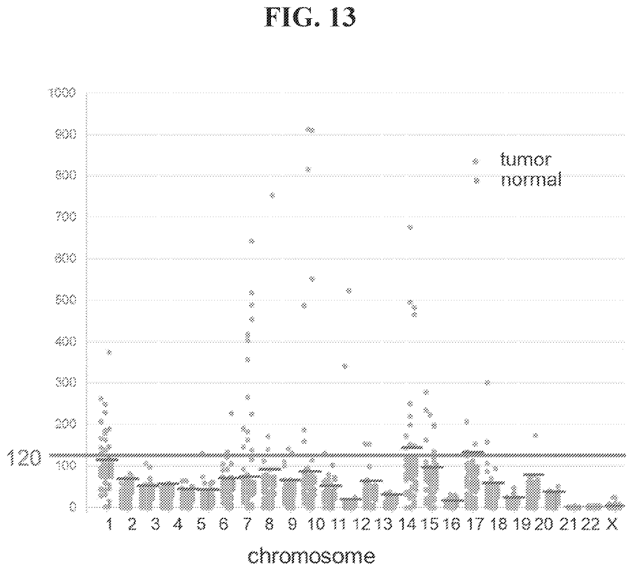

As shown in FIG. 11, all stage I tumors were GAPF-positive. FIG. 12 shows subtype-specific distribution of high coverage bins between Luminal A and triple-negative breast cancer (TNBC). See chromosome 11 in FIG. 12. FIG. 13 shows a chromosome-specific threshold, and in this case, if any one of the chromosomes has more than 120 bins out of top 1,000 bins, the DNA is from tumor, and thus, is GAPF-positive.

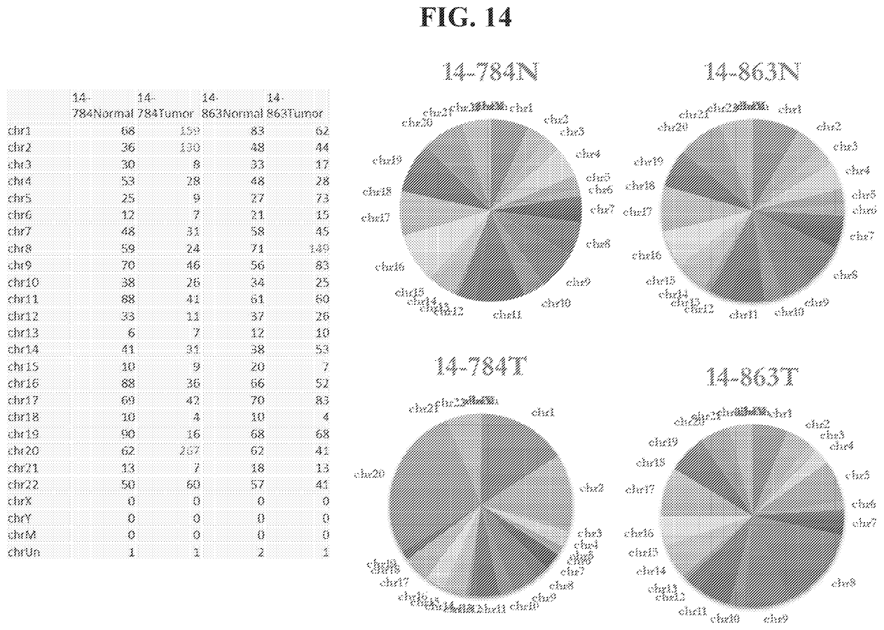

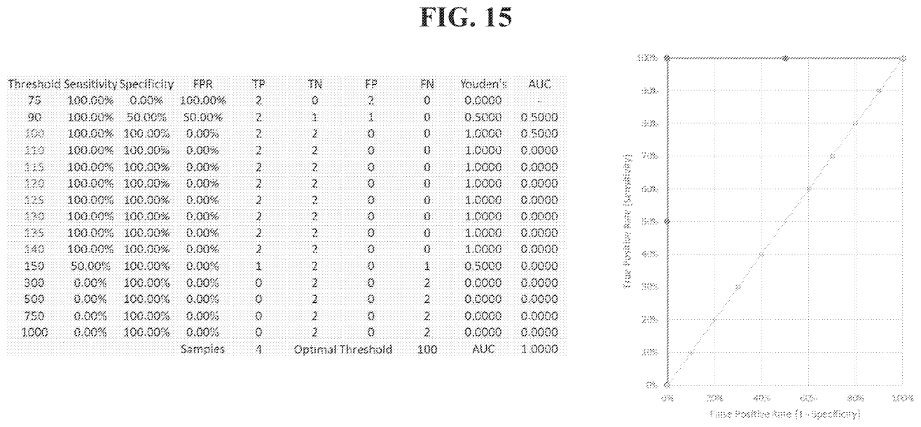

FIG. 14 shows the distributions of top 1000 1-kb bins of GAPF-seq data in two lung tumor/normal pairs, indicating that GAPF-seq data could effectively differentiate between two lung tumor/normal pairs. According to FIG. 14, the distributions of bins were skewed in both tumors (>100 bins/chromosome). Referring to FIG. 15 showing an ROC curve and bin threshold estimate from the top 1000 1-kb bins data from two lung normal/tumor pairs, the numbers of GAPF-seq reads were counted for 1-kb bins throughout the genome (approximately 3 million bins), and the top 1000 bins were taken for the analysis.

Although above discussed studies of GAPF were done using breast/lung cancer tissue biopsy, circulating tumor DNA detection in liquid biopsy would solve a “needle in a haystack” problem for application of GAPF. The following describes another study using liquid biopsy. Liquid biopsy can potentially include major cancer types including breast, prostate, and lung cancer among others and even minor ones.

Invasive tumors release DNA, RNA, and proteins into body fluids. For example, the body fluids include interstitial fluid, intravascular fluid, transcellular fluid, amniotic fluid, aqueous humor, bile, blood, whole blood, blood serum, blood plasma, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. For example, the body fluids include blood. For example, the body fluids include blood plasma.

These biomolecules, such as DNA, RNA, and proteins, are alternative sources for cancer detection and monitoring (liquid biopsy). Liquid biopsy can be a more cost-effective and less invasive approach for the diagnosis and monitoring of cancer patients than currently available measures at the clinic (such as needle biopsies or imaging scans). The global liquid biopsy market size accounted for $1.2 billion in 2020 and is expected to undergo continuous and rapid growth, reaching $6.8 billion by 2028. Blood has been a source of protein biomarkers of cancer such as Prostate-specific Antigen (PSA) and Carcinoembryonic Antigen (CEA) With the advancement of sequencing technologies, tumor-derived DNA in blood (circulating tumor DNA, ctDNA) has become a primary target for cancer detection.

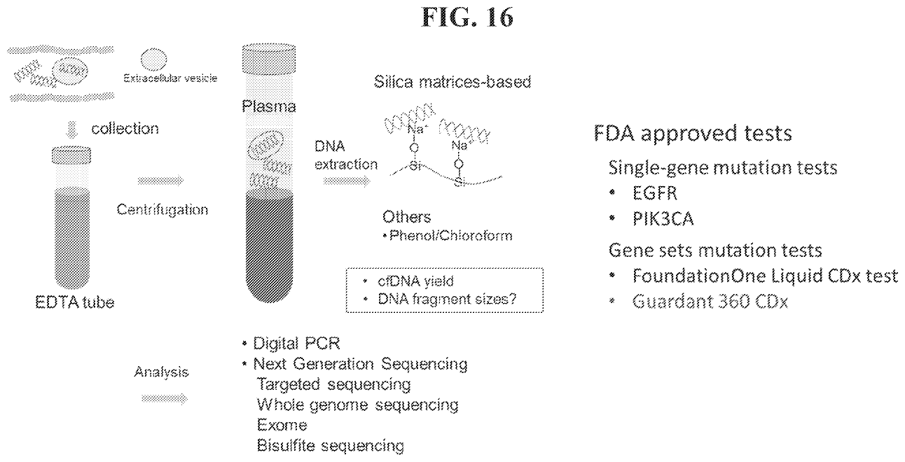

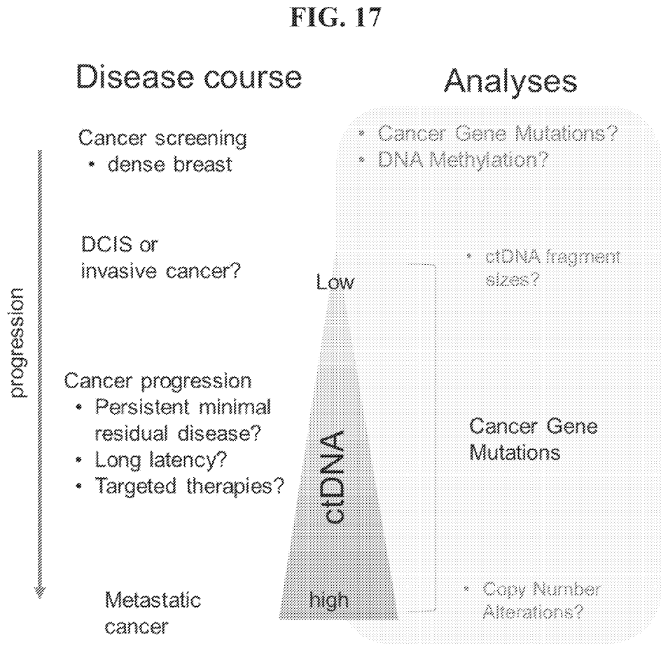

FIG. 16 shows an exemplary plasma DNA analysis which is a mainstream of liquid biopsy. FIG. 17 illustrates liquid biopsy and breast cancer progression.

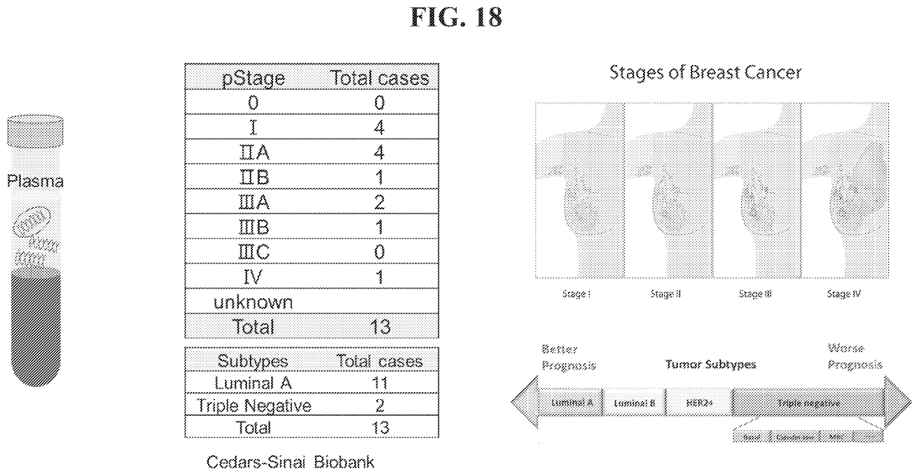

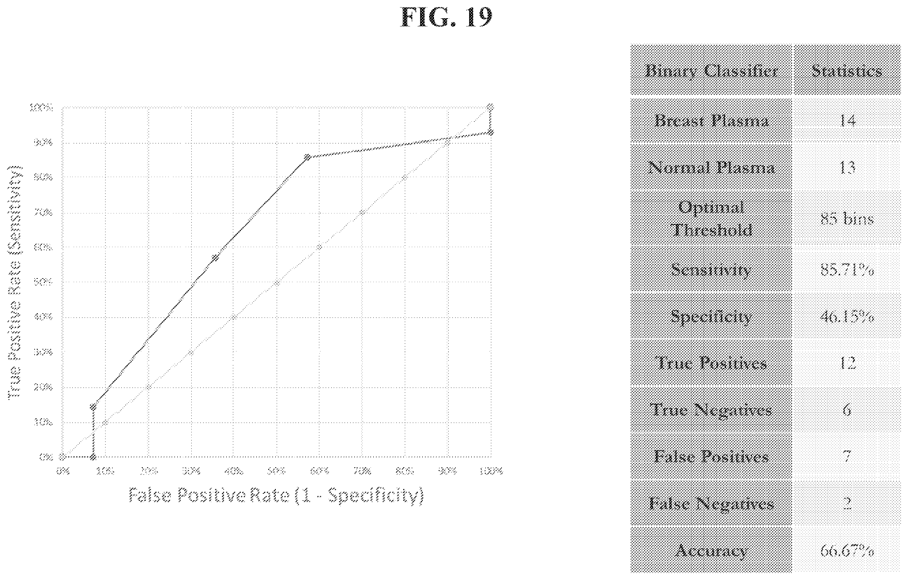

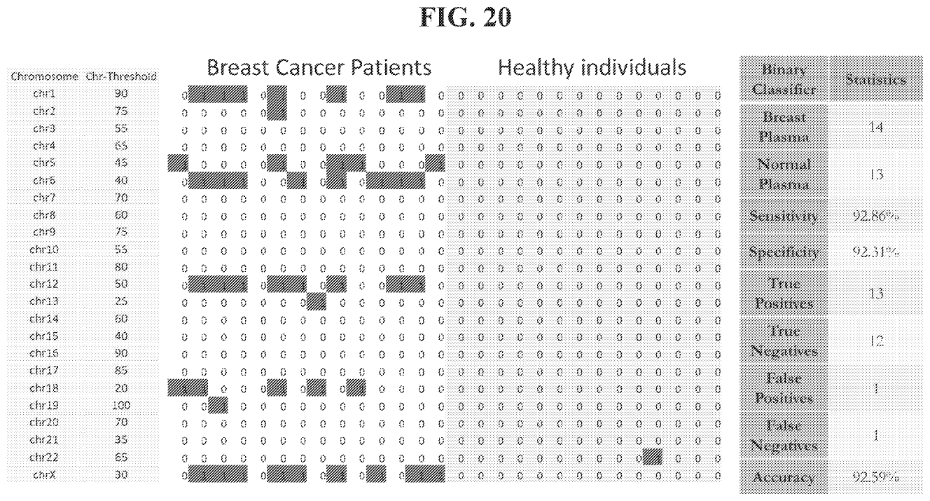

FIG. 18 shows distinguishing breast tumor DNA from normal DNA by GAPF-seq in liquid biopsy. FIG. 19 shows an ROC curve for breast plasma/normal plasma classifier, i.e., a binary classifier based on a genome-wide threshold. FIG. 20 shows a binary classifier based on a chromosome-specific threshold. Compared to 66.7% accuracy, 85.71% sensitivity, and 46.15% specificity of the genome-wide threshold, 92.59% accuracy, 92.86% sensitivity, and 92.31% specificity of the chromosome-specific threshold is promising, and it shows that GAPF-seq can be applied to liquid biopsy.

### EXAMPLES

### Example 1

Here, we describe a modified GAPF protocol for isolating and amplifying DNA palindromes from genomic DNA sources with low input DNA amounts and a bioinformatics pipeline for assessing the enrichment and location of de novo palindrome formation. Native DNA palindromes typically represent a structural challenge for genomic studies because the Taq polymerase involved in PCR and library construction for whole genome sequencing cannot navigate the secondary structure of self-annealed palindromes. Therefore, these technologies may underestimate palindromes and fold-back inversions. With GAPF, the denaturing and renaturing step prior to any PCR steps converts the DNA palindrome into dsDNA amenable to amplification by PCR. Furthermore, this procedure for enriching palindromes confers the advantages of simultaneously amplifying target signal (via PCR) and reducing background noise (via S1 nuclease digestion) without targeted analysis and thus, can efficiently present palindromes in sequencing data without ultra-deep sequencing.

### Materials

### GAPF

1. SbfI-HF and CutSmart buffer (New England Biolabs, R3642S)
2. KpnI-HF and NEBuffer 1.1 (New England Biolabs, R3142S)
3. S1 nuclease and buffer (Invitrogen, 18001016)
4. Monarch PCR & DNA Clean-up Kit (New England Biolabs, T1030S)
5. Formamide
6. 5M NaCl
7. Nuclease-free H₂O
8. Microcentrifuge tubes
9. Thin-wall microcentrifuge tubes
10. Microcentrifuge tube cap locks.

### Library Construction

1. NEBNext Ultra II FS DNA Library Prep Kit for Illumina (New England Biolabs, E7805S)
2. AMPure XP Beads (Beckman Coulter Inc., A63881)
3.10 mM Tris-HCl, pH 7.5-8.0 with 10 mM NaCl (for adapter dilution)
4. 80% ethanol (freshly prepared)
5. 1× TE: 10 mM Tris, pH 8.0, 1 mM EDTA
6. 0.1× TE: 1:10 dilution of 1× TE in water
7. Qubit Assay Kit (Invitrogen, Q32851)
8. Thin-wall PCR tube strips
9. Magnetic stand/rack.

### Methods

Prepare all solutions using analytical grade reagents and store them at room temperature unless indicated otherwise. Carry out all procedures at room temperature unless specified otherwise. Follow waste disposal regulations when disposing waste materials.

### DNA Fragmentation (Restriction Enzyme Digestion)

1. Mix 30-1,000 ng of DNA with nuclease-free H₂O to a total volume of 34 μL in a 1.7 mL microcentrifuge tube. This protocol has been optimized to efficiently enrich palindromes from low input DNA sources.
2. In a new 1.7 mL microcentrifuge tube, mix 17 μL of the DNA solution with 1 μL KpnI (10 U), and 2 μL 10× NEBuffer 1.1 for a total volume of 20 μL. Digestion by restriction enzymes is necessary to cut DNA into fragments that can be effectively denatured by boiling in later steps.
3. In a new 1.7 mL microcentrifuge tube, mix the remaining 17 μL of the DNA solution with 1 μL SbfI (10 U), and 2 μL CutSmart buffer for a total volume of 20 μL. In order to capture large DNA palindromes (˜5 kb), DNA needs to be cut infrequently by KpnI or SbfI and so, GAPF performs best when these restriction enzymes are used separately.
4. Incubate at 37° C. in a water bath overnight (>16 hr).
5. Briefly spin in a microcentrifuge to bring the liquid to the bottom.
6. Heat at 65° C. for 20 min to inactivate restriction enzymes.

### Snap-Back

1. Briefly spin in a microcentrifuge to bring the liquid to the bottom.
2. Mix the 20 μL of KpnI-digested DNA and 20 μL of SbfI-digested DNA with 1.8 μL 5M NaCl, 45 μL formamide, and 3.2 μL nuclease-free H₂O in a thin-wall PCR tube. Minimize the amount of time that KpnI and SbfI are combined prior to boiling. Formamide and NaCl are necessary additions in order to facilitate efficient DNA denaturing.
3. Apply a cap lock to prevent the tube from opening during DNA denaturing.
4. Heat the DNA mixture in boiling water for 7 min to denature DNA.
5. Immediately quench the DNA mixture in ice water for 5 min to rapidly renature DNA.

### S1 Digestion

1. Briefly spin in a microcentrifuge to bring the liquid to the bottom.
2. Add 4.8 μL 5M NaCl, 12 μL 10× S1 nuclease buffer, 2 μL S1 nuclease (20 U/μL), and 11.2 μL nuclease-free H₂O to the DNA mixture.
3. Incubate at 37° C. in a water bath for 1 hr.

### Purify DNA (Monarch PCR & DNA Clean-up Kit)

1. Centrifugation should be carried out at 16,000×g (˜13,000 rpm) at room

temperature.
2. Add 240 μL DNA Cleanup Binding Buffer to the S1 digested-DNA sample.
3. Mix well by pipetting 10 times.
4. Briefly spin in a microcentrifuge to bring the liquid to the bottom.
5. Move liquid to a column, insert column into a 2 mL collection tube, and close the cap.
6. Centrifuge for 1 min and then discard the flow-through.
7. Add 200 μL DNA Wash Buffer, centrifuge for 1 min, and then discard the flow-through.
8. Repeat step 7 once.
9. Insert the empty column into the collection tube and centrifuge for 1 min.
10. Transfer the column to a new collection tube.
11. Add 15 μL DNA Elution Buffer and incubate for 1 min at room temperature.
12. Centrifuge for 1 min.
13. Add 10 μL DNA Elution Buffer and incubate for 1 min at room temperature.
14. Centrifuge for 1 min and save the sample. 2 or 3 μL of sample can be used for measuring sample concentration by a Qubit 3.0 Fluorometer to assess the overall depletion

### Library Construction (NEBNext Ultra II FS DNA Library Prep Kit for Illumina)

1. Mix 22 μL of DNA, 4 μL nuclease-free H₂O, 7 μL NEBNext Ultra II FS Reaction Buffer, and 2 μL NEBNext Ultra II FS Enzyme Mix in a PCR tube.
2. Vortex reaction for 5 sec and briefly spin in a centrifuge to bring the liquid to the bottom.
3. In a thermocycler with the lid heated to 75° C., incubate the reaction for 15 min at 37° C. followed by 30 min at 65° C. and then held at 4° C.
4. Add to the reaction mixture 1 μL Ligation Enhancer, 2.5 μL diluted NEBNext Adaptor, and 30 μL Ligation Master Mix.
5. Mix well by pipetting 10 times set to 50 μL and briefly spin in a microcentrifuge to bring the liquid to the bottom.
6. In a thermocycler with no heated lid, incubate the reaction for 15 min at 20° C. and then held at 4° C.
7. Add to the reaction mixture 3 μL USER Enzyme.
8. Mix well by pipetting 10 times set to 50 μL and briefly spin in a microcentrifuge to bring the liquid to the bottom.
9. In a thermocycler with the lid heated to at least 47° C., incubate the reaction for 15 min at 37° C. and then held at 4° C.
10. Vortex magnetic beads
11. Add 57 μL magnetic beads to adaptor-ligated DNA.
12. Incubate at room temperature for 5 min.
13. Place magnetic bead-DNA mixture on magnet for 5 min.
14. Remove supernatant.
15. On magnet, add 200 μL 80% ethanol, wait for 30 sec, and then remove supernatant.
16. Repeat step 15 once.
17. Air dry the magnetic beads for 3 min.
18. Off magnet, add 17 μL 0.1× low TE buffer.
19. Mix well by pipetting 10 times.
20. Incubate at room temperature for 5 min.
21. Place magnetic bead-DNA mixture on magnet for 5 min.
22. Remove 15 μL supernatant and put into a new PCR tube.
23. Add 5 μL Universal PCR Primer, 5 μL Index Primer, and 25 μL NEBNext Q5 Master Mix.
24. Mix well by pipetting 10 times set to 40 μL and briefly spin in a microcentrifuge to bring the liquid to the bottom.
25. In a thermocycler with the lid heated to at least 103° C., incubate the reaction for 30 sec at 98° C. followed by 20 cycles of 10 sec at 98° C. and 75 sec at 65° C., then 5 min at 65° C. and held at 4° C.
26. Vortex magnetic beads.
27. Add 45 μL magnetic beads to adaptor-ligated DNA.
28. Incubate at room temperature for 5 min.
29. Place magnetic bead-DNA mixture on magnet for 5 min.
30. Remove supernatant.
31. On magnet, add 200 μL 80% ethanol, wait for 30 sec, and then remove supernatant.
32. Repeat step 31 once.
33. Air dry the magnetic beads for 3 min.
34. Off magnet, add 33 μL 0.1× low TE buffer.
35. Mix well by pipetting 10 times.
36. Incubate at room temperature for 5 min.
37. Place magnetic bead-DNA mixture on magnet for 5 min.
38. Remove 30 μL supernatant and store in a DNA LoBind tube.
39. Measure concentration of DNA with High Sensitivity Qubit Fluorometer for dsDNA using 2 μL of sample.
40. Check size distribution with Agilent Bioanalyzer High Sensitivity DNA chip.
41. Sequence samples using an Illumina-based sequencing platform with low sequencing depth (0.5-1.0× coverage is sufficient).

### Data Analysis

1. Trim raw *.fastq data with Trim_galore (v0.6.1) and Cutadapt (v2.3) with parameters ‘--length 55’.
2. Align trimmed *.fastq data to hg38 reference genome using Bowtie2 (v2.3.5) with unpaired alignment.
3. Convert *.sam alignment file using Samtools (v1.9) to binary format and sort the subsequent *.bam files.
4. Filter uniquely mapped reads by applying a mapping quality filter of 40 μsing the ‘samtools view’ command with parameters ‘-b -q 40’. The hg38 human reference genome contains palindromic sequences that will be amplified by this procedure. Because alignment software will attempt to find a single point of origin, reads that can align to either arm of the palindrome will have a low mapping quality. To detect de novo palindrome formation in tumor samples, palindromes found in the reference genome can be removed using a filter for uniquely mapped reads.
5. Extract the number of sequencing reads after applying the mapped quality filter to determine the per million scaling factor to normalize for mapping depth. The per-million scaling factor is calculated by dividing the total number of reads in the file by 1,000,000.
6. Sort *.bam file using Samtools and convert to *.bed format using Bedtools (v2.28.0).
7. Sort *.bed files using the ‘sort’ command with parameters ‘-k1, 1-k2,2n’.
8. Use Bedtools2 to take an alignment of reads as input and generate a coverage track as output in 1 kb non-overlapping bins with parameters ‘-sorted-counts’.
9. Use the scaling factor to normalize the coverage in 1 kb bins for the mapping depth.
10. Locate regions of high coverage bins to identify de novo DNA palindromes. The threshold for what is considered “high coverage” can change depending on how efficiently GAPF enriched DNA palindromes. After the per-million scaling factor, the average coverage in 1 kb bins is approximately 0.3, so an appropriate threshold may be between 1.0 and 5.0 depending on the background signal in single-copy regions of the genome.

### Example 2

### Cancer Detection in Plasma Cell-Free DNA From Prostate Cancer Patients by GAPF-Seq

Cancer detection in plasma cfDNA has a significant impact on the management of cancer patients and screening in the general population. Chromosomal aberrations are the manifestations of cancer; however, currently available methods lack the sensitivity for small tumor fractions in cancer patients' plasma cfDNA. Given the potentially improved sensitivity, GAPF-seq would be a powerful approach for the detection of cfDNA.

We tested the utility of GAPF-seq for cancer detection using 10 samples of plasma cfDNA from prostate cancer patients. DNA was extracted from plasma and buffy coats from each patient and treated by GAPF-seq protocol. In addition, shallow WGS (100 million 150 bp-reads/sample, 0.5× genome coverage) was conducted for plasma cfDNA and determined the tumor fraction by ichorCNA. ichorCNA quantifies tumor contents in cfDNA from shallow WGS data and has been widely used to evaluate ctDNA fraction in cfDNA. Among the 10 samples, tumor fraction was estimated to be >0.1 for 5 samples, the cutoff for calling the presence of tumor-derived DNA with high sensitivity (0.91).

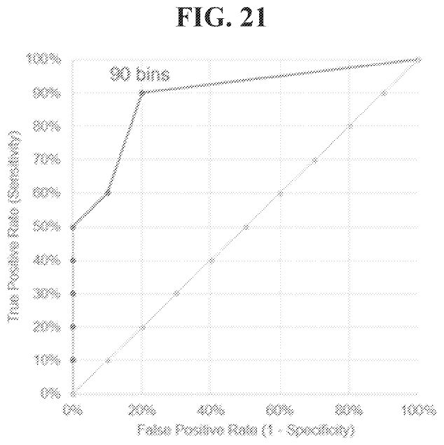

Using the number of top 1000 bins in each chromosome as the binary classification rule, we generated the ROC curve and found that AUC was 0.89. See FIG. 21. With the 90-bins threshold, sensitivity was 90%, and specificity was 80%.

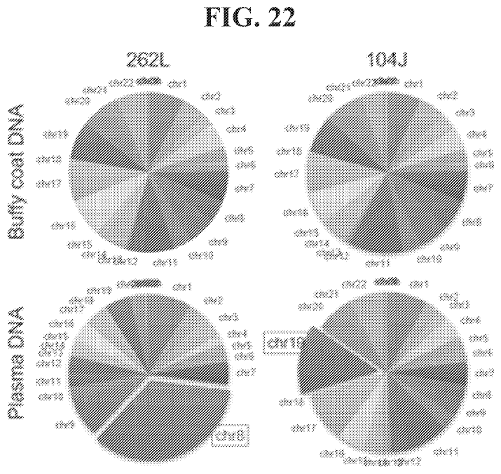

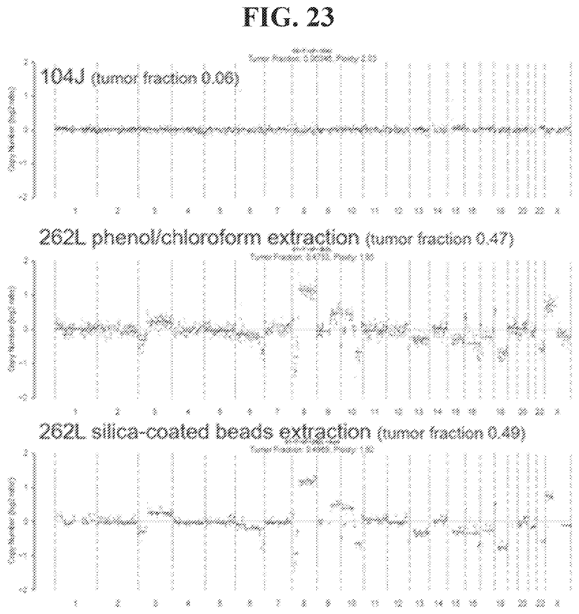

Skewed distributions of the top 1000 bins were shown in FIG. 22, in which bins were overrepresented in chr19 (104J) and chr8 (262L) from plasma DNA GAPF-profiles. Importantly, tumor fraction was low, less than 0.1 in 104J plasma DNA (FIG. 23 top).

### Example 3

### Use of Machine Learning Algorithms to Validate Previous Results Including ROC Curves

ROC curves discussed above, for example as shown in FIGS. 5A, 5B, 10-14, 21-23, were drawn manually. Such ROC curves can be validated using machine learning algorithms. For example, multiple machine learning algorithms are applied to validate the superb performances of GAPF profiles in separating tumor and cfDNA from matched normal DNA (from leukocytes).

In earlier discussion above, we presented manually-drawn ROC curves and showed the high performance of GAPF-seq and profiles in separating tumor and cfDNA from paired normal DNA. We applied machine learning approaches to our GAPF-seq data and tested the performance of the data for binary classification (tumor DNA and normal DNA). We employed automated machine learning pipeline Streamline (doi.org/10.48550/arXiv.2206.12002). Streamline is designed to evaluate the performance of various machine learning algorithms. The input dataset will be partitioned into three groups, with two groups combined for training the algorithms to develop models and the remaining as a test set for evaluation. This three-fold cross-validation of training and test sets will assess the algorithm's predictive performance and flag potential problems such as overfitting or selection bias.

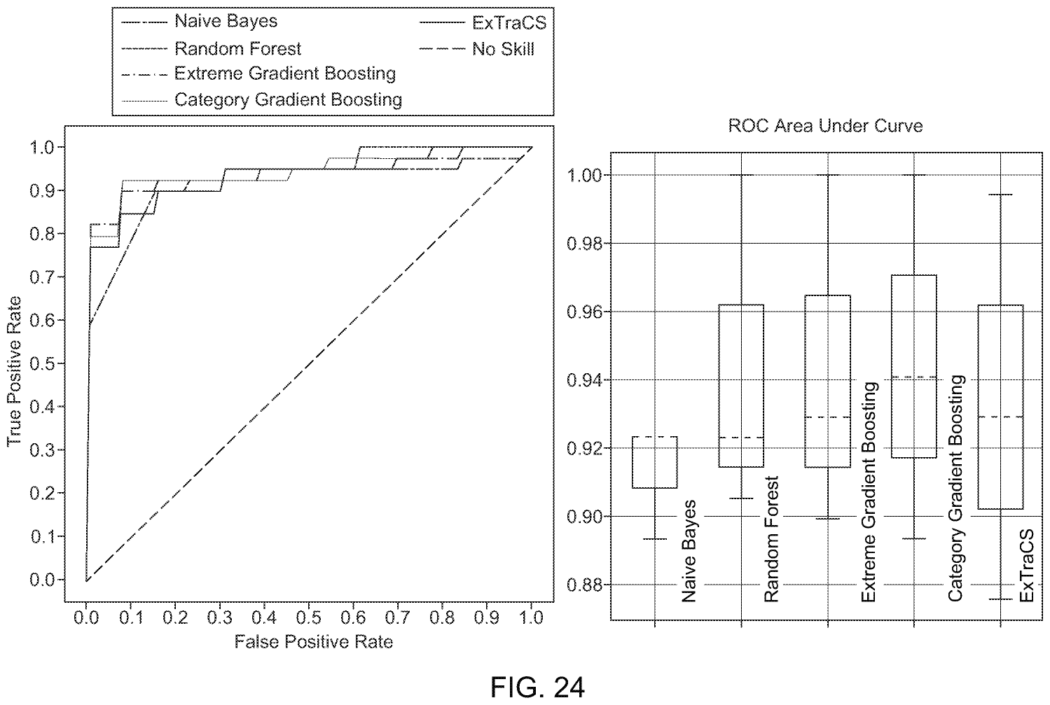

GAPF profiles show high performance in binary classification between tumor DNA and normal DNA. We input the numbers of the top 1000 bins in each chromosome (1-22 and X) into the pipeline and evaluated the performance using five algorithms. From the 39 pairs of breast tumor and matched normal leukocyte DNA, the average ROC AUC values were consistently very high across machine learning algorithms (FIG. 24, left). Although there were variances between different partitions of test sets, all five algorithms scored >0.9 (FIG. 24, right).

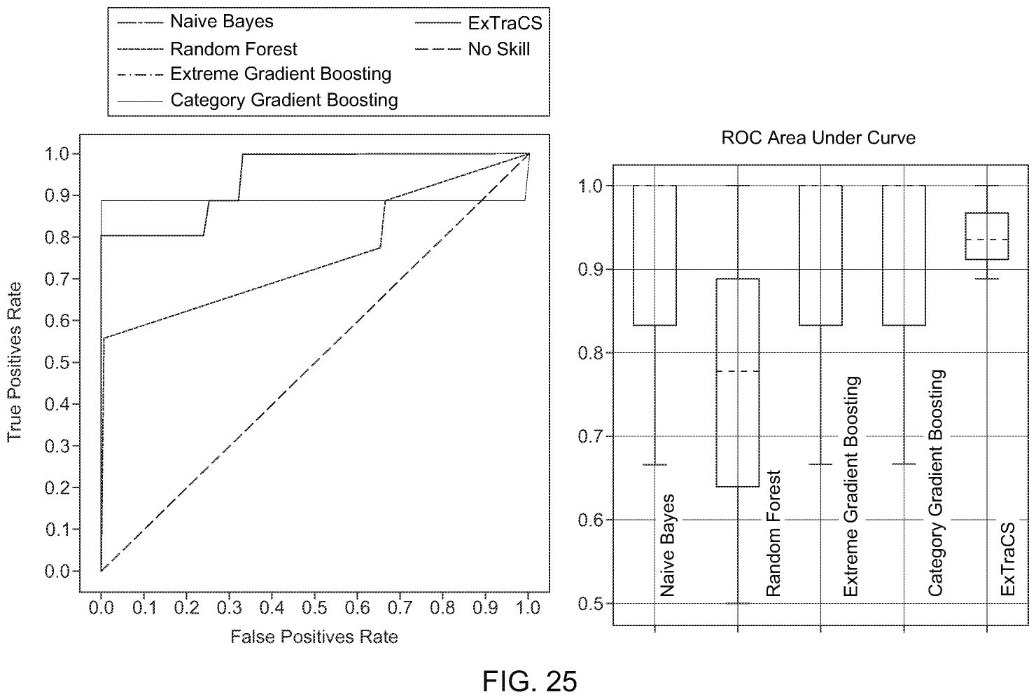

Referring to FIG. 25, GAPF-profiles from prostate cfDNA also scored very high average AUC (0.942 by ExTraCS, 0.889 by Naïve Bayes and the other two algorithms, and 0.759 by Random Forest). Despite the low sample size, the variance in AUC between cross-validation iterations was low, especially for ExTraCS. Therefore, this approach shows enough promise to warrant further exploration with larger sample sizes.

GAPF-seq requires high molecular weight DNA (HMW DNA). Tumor fractions could be more abundant in very short DNA fragments in cfDNA extracted by a commercially available kit, although the biological ground for the observation remains elusive. Also, no one has compared tumor fraction between commercially available kits-extracted and phenol chloroform-extracted plasma cfDNA. To test the feasibility of HMW DNA for cancer detection, we extracted DNA from the plasma (262L) using either phenol/chloroform approach or silica-coated beads (Apostle Kit, Beckman). Shallow WGS with ichorCNA was used to quantify tumor fraction. Both genome-wide copy number profiles and tumor fractions were comparable between two DNA samples (FIG. 23, middle and bottom). Overall, these data suggest that GAPF-seq is feasible and could detect tumor DNA in patients' plasma.

We have shown that GAPF-profiles, produced from GAPF-seq, can differentiate tumor DNA from normal DNA with very high sensitivity and specificity. GAPF-profiles were reproducible. GAPF-profiles can be breast tumor subtype-specific. We extended GAPF-seq to cell-free DNA from cancer patients' plasma. GAPF-seq could distinguish cancer patients' cfDNA from normal DNA even when the tumor fraction was very low. Because DNA palindrome formation is an initial step of genomic amplification, we envision that GAPF-profiles could capture genomic changes at the early stage of oncogene or therapy resistance gene amplification.

Because of the association with cancer genome instability, DNA palindromes are expected to occur commonly and to have substantial implications in a variety of tumors. GAPF-seq could serve as a genomic test for pan-cancer detection and risk assessment. For example, the following cancers can be detected among others by the genomic test: breast cancer, lung cancer, prostate cancer, bladder cancer, cervical cancer, colorectal cancer, gynecologic cancers including cervical, ovarian, uterine, vaginal, and vulvar, head and neck cancers, kidney cancer, liver cancer, lymphoma, mesothelioma, myeloma, ovarian cancer, skin cancer, thyroid cancer, uterine cancer, and vaginal and vulvar cancers.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method of detecting invasive tumor in a subject, the method comprising:
denaturing genomic DNA isolated from a tumor sample obtained from the subject or denaturing cell-free DNA (cfDNA) isolated from a body fluid obtained from the subject having the tumor, to generate denatured DNA;
renaturing the denatured DNA for tumor DNA palindrome to form a snap back DNA;
digesting the renatured DNA with a nuclease that digests single strand DNA;
amplifying the tumor DNA palindrome by adapter ligation-mediated polymerase chain reaction (PCR) with genome-wide analysis of Palindrome Formation (GAPF);
performing a sequence scan across multiple samples of the amplified tumor DNA palindrome;
mapping reads of GAPF-seq from the sequence scan into a plurality of bins;
quantifying reads in each bin; and
determining whether the tumor sample is an invasive tumor based on presence of tumor-derived DNA in the genomic DNA or cfDNA and/or GAPF profiles generated by analyzing the quantified reads in each bin.

2. The method of claim 1, wherein:
the subject is determined to have the invasive tumor when the tumor sample or body fluid is GAPF-positive or when any one of chromosomes has more than a threshold number of bins out of top 1,000 bins; or
the subject is determined to not have an invasive tumor when the tumor sample or body fluid is GAPF-negative or when no tumor DNA palindromes are detected from the isolated genomic DNA or cfDNA.

3. The method of claim 2, wherein the determination is based on a chromosome-specific threshold.

4. The method of claim 1, wherein the tumor is stage I tumor or luminal A tumor.

5. (canceled)

6. The method of claim 4, wherein the tumor DNA palindrome clusters at CCND1 oncogene loci in the luminal A tumor.

7. The method of claim 1, wherein the subject has breast cancer or lung cancer.

8. (canceled)

9. The method of claim 1, wherein numbers of the GAPF-seq reads are counted for 1-kb bins.

10. The method of claim 9, wherein top 1,000 bins are taken for analysis to determine the presence of the invasive tumor.

11. The method of claim 1, wherein the body fluid of the subject comprises interstitial fluid, intravascular fluid, transcellular fluid, amniotic fluid, aqueous humor, bile, blood, whole blood, blood serum, blood plasma, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit.

12. The method of claim 11, wherein the body fluid comprises blood or blood plasma.

13. The method of claim 1, wherein the sequence scan is a shallow scan, and the method does not require deep sequencing.

14. The method of claim 1, wherein an amount of the genomic DNA required for generation of the GAPF profiles is about 10 ng-about 50 ng, about 20 ng-about 40 ng, about 25 ng-about 35 ng, or about 30 ng or less.

15. (canceled)

16. (canceled)

17. (canceled)

18. The method of claim 1, further comprising isolating the genomic DNA from the tumor before denaturing the genomic DNA.

19. The method of claim 1, further comprising isolating the cf DNA from the body fluid before denaturing the cfDNA.

20. (canceled)

21. A method of treating a subject with cancer based on invasiveness of tumor, the method comprising:
administering a treatment to the subject, the treatment comprising biopsy, surgery, chemotherapy, hormone therapy, and/or radiation therapy if the tumor is an invasive tumor, or the treatment comprising active monitoring, not performing biopsy, surgery, chemotherapy, hormone therapy, and radiation therapy on the subject, if the tumor is not an invasive tumor,
wherein tumor invasiveness is detected by:
denaturing genomic DNA isolated from a tumor sample obtained from the subject or denaturing cell-free DNA (cfDNA) isolated from body fluid obtained from the subject having the tumor, to generate denatured DNA;
renaturing the denatured DNA for tumor DNA palindrome to form a snap back DNA;
digesting the renatured DNA with a nuclease that digests single strand DNA;
amplifying the tumor DNA palindrome by adapter ligation-mediated polymerase chain reaction (PCR) with genome-wide analysis of Palindrome Formation (GAPF);
performing a sequence scan across multiple samples of the amplified tumor DNA palindrome;
mapping reads of GAPF-seq from the sequence scan into a plurality of bins;
quantifying reads in each bin; and
determining the invasiveness of the tumor in the subject based on presence of tumor-derived DNA in the genomic DNA or cfDNA and/or GAPF profiles generated by analyzing the quantified reads in each bin.

22. The method of claim 21, wherein the cancer comprises breast, prostate, or lung cancer.

23. The method of claim 22, wherein the treatment of the breast cancer comprises surgery, radiation, chemotherapy, hormone therapy, targeted drug therapy, and/or immunotherapy based on a stage/type of the breast cancer if the tumor is an invasive tumor.

24. The method of claim 22, wherein the treatment of the lung cancer comprises surgery, chemotherapy, radiation therapy, targeted drug therapy, immunotherapy, palliative care, and/or alternative medicine such as acupuncture, hypnosis, massage, meditation, and yoga based on a stage/type of the lung cancer if the tumor is an invasive tumor.

25. The method of claim 22, wherein the treatment of the prostate cancer comprises surgery, radiation, cryotherapy, hormone therapy, chemotherapy, immunotherapy, and/or targeted drug therapy based on a stage/type of the prostate cancer if the tumor is an invasive tumor.

26. The method of claim 21, wherein the body fluid of the subject comprises interstitial fluid, intravascular fluid, transcellular fluid, amniotic fluid, aqueous humor, bile, blood, whole blood, blood serum, blood plasma, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit.

27. The method of claim 26, wherein the body fluid comprises blood or blood plasma.

28. (canceled)
